SEQUENCE LISTING (52 sequences)

SEQ ID NO: 1 - protein, 533 aa, Homo sapiens
MWLGRRALCALVLLLACASLGLLYASTRDAPGLRLPLAPWAPPQSPRRPELPDLAPEPRYAHIPVRIKEQVVGLLAWNNCSCESSGGGLPLPFQKQVRAIDLTKAFDPAELRAASATREQEFQAFLSRSQSPADQLLIAPANSPLQYPLQGVEVQPLRSILVPGLSLQAASGQEVYQVNLTASLGTWDVAGEVTGVTLTGEGQADLTLVSPGLDQLNRQLQLVTYSSRSYQTNTADTVRFSTEGHEAAFTIRIRHPPNPRLYPPGSLPQGAQYNISALVTIATKTFLRYDRLRALITSIRRFYPTVTVVIADDSDKPERVSGPYVEHYLMPFGKGWFAGRNLAVSQVTTKYVLWVDDDFVFTARTRLERLVDVLERTPLDLVGGAVREISGFATTYRQLLSVEPGAPGLGNCLRQRRGFHHELVGFPGCVVTDGVVNFFLARTDKVREVGFDPRLSRVAHLEFFLDGLGSLRVGSCSDVVVDHASKLKLPWTSRDAGAETYARYRYPGSLDESQMAKHRLLFFKHRLQCMTSQ

SEQ ID NO: 2 - protein, 113 aa, Artificial sequence [sequence omitted]

SEQ ID NO: 3 - protein, 113 aa, Artificial sequence
  Anti-GD2 hu14.18 VH
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSS

SEQ ID NO: 4 - protein, 108 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 5 - protein, 113 aa, Artificial sequence [sequence omitted]

SEQ ID NO: 6 - protein, 113 aa, Artificial sequence
  Anti-GD2 hu14.18 VL
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 7 - protein, 107 aa, Homo sapiens [sequence omitted]

SEQ ID NO: 8 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 42 aa, Homo sapiens
PRKIEVMYPPPYLDNEKSNGTIIHVKGKHLCPSPLFPGPSKP

SEQ ID NO: 10 - protein, 48 aa, Homo sapiens
AKPTTTPAPRPPTPAPTIASQPLSLRPEACRPAAGGAVHTRGLDFAPR

SEQ ID NO: 11 - protein, 88 aa, Artificial sequence
  CD8 hinge + CD28 hinge (long hinge)
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 12 - protein, 27 aa, Homo sapiens
FWVLVVVGGVLACYSLLVTVAFIIFWV

SEQ ID NO: 13 - protein, 23 aa, Homo sapiens
IYIWAPLAGTCGVLLLSLVITLY

SEQ ID NO: 14 - protein, 27 aa, Homo sapiens
IISFFLALTSTALLFLLFFLTLRFSVV

SEQ ID NO: 15 - protein, 21 aa, Homo sapiens
LCYLLDGILFIYGVILTALFL

SEQ ID NO: 16 - protein, 42 aa, Homo sapiens
KRGRKKLLYIFKQPFMRPVQTTQEEDGCSCRFPEEEEGGCEL

SEQ ID NO: 17 - protein, 41 aa, Homo sapiens
RSKRSRLLHSDYMNMTPRRPGPTRKHYQPYAPPRDFAAYRS

SEQ ID NO: 18 - protein, 42 aa, Homo sapiens
ALYLLRRDQRLPPDAHKPPGGGSFRTPIQEEQADAHSTLAKI

SEQ ID NO: 19 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 38 aa, Homo sapiens
RVKFSRSADAPAYQQGQNQLYNELNLGRREEYDVLDKR

SEQ ID NO: 21 - protein, 47 aa, Homo sapiens
RVKFSRSADRGRDPEMGGKPRRKNPQEGLYNELQKDKMAEAYSEIGM

SEQ ID NO: 22 - protein, 45 aa, Homo sapiens
RVKFSRSADKGERRRGKGHDGLYQGLSTATKDTYDALHMQALPPR

SEQ ID NO: 23 - protein, 19 aa, Mus musculus
MEWSWVFLFFLSVTTGVHS

SEQ ID NO: 24 - protein, 20 aa, Mus musculus
MSVPTQVLGLLLLWLTDARC

SEQ ID NO: 25 - protein, 21 aa, Mus musculus
MALPVTALLLPLALLLHAARP

SEQ ID NO: 26 - protein, 21 aa, Artificial sequence
  T2A self-cleaving sequence
GSGEGRGSLLTCGDVEENPGP

SEQ ID NO: 27 - protein, 22 aa, Artificial sequence
  P2A self-cleaving sequence
GSGATNFSLLKQAGDVEENPGP

SEQ ID NO: 28 - protein, 23 aa, Artificial sequence
  E2A self-cleaving sequence
GSGQCTNYALLKLAGDVESNPGP

SEQ ID NO: 29 - protein, 25 aa, Artificial sequence
  F2A self-cleaving sequence
GSGVKQTLNFDLLKLAGDVESNPGP

SEQ ID NO: 30 - protein, 724 aa, Artificial sequence
  Precursor  GD2-14.18 DAR (version V2a)
MEWSWVFLFFLSVTTGVHSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTPRKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 2172 bp, Artificial sequence
  GD2-14.18 DAR precursor
atggaatggagctgggtgttcctcttcttcctcagcgtgaccaccggcgtgcactccgaggtgcaattattacagtccggacccgagctggagaagcccggcgcctccgtgatgatcagctgtaaagcctccggcagctcctttaccggctacaacatgaactgggtgaggcagaacattggaaagtctttagagtggatcggcgccatcgatccctactacggcggcacctcctataaccagaagttcaagggtcgtgccaccctcaccgtcgacaagtcctccagcacagcttatatgcatttaaagtccctcaccagcgaggactccgccgtgtattattgtgtgagcggcatggaatattggggccaaggaaccagcgtcaccgtgtccagcgcttccacaaagggacccagcgtcttccctctggcccccagcagcaaaagcaccagcggcggaaccgctgctttaggatgtttagtgaaggactactttcccgaacccgtgaccgtgagctggaattccggcgctctcaccagcggcgtgcacacctttcccgctgtcctccagtccagcggcctctactctttatcctccgtggtgacagtgcctagctcctccctcggcacccagacctacatttgtaacgtgaaccacaagccttccaatacaaaggtggacaagagggtggagcccaaaagctgcgataagacccacactcctcgtaagattgaggtcatgtacccccccccctatctggacaacgagaagagcaacggcacaatcatccacgtgaagggcaagcatctgtgcccctcccctttatttcccggaccttccaaacccttttgggttttagtggtcgtgggaggcgtgctggcttgttactctttactggtgacagtcgccttcatcatcttttgggtgaagaggggtcgtaagaaactgctgtacatcttcaaacagcccttcatgaggcccgtgcagacaacccaagaagaggatggatgttcttgtcgtttccccgaagaggaggagggaggctgtgaactgagggtgaagttctctcgtagcgctgacgcccccgcctaccaacaaggtcaaaaccagttatacaacgagctgaatttaggaaggagagaggagtatgacgttttagacaagagaaggggaagagaccccgaaatgggcggcaagcccagaaggaagaacccccaagaaggtttatacaatgagctccagaaggacaagatggccgaggcctacagcgaaattggcatgaaaggcgagaggaggaggggaaagggccatgacggcctctatcaaggtttaagcaccgccaccaaggacacctacgacgctttacatatgcaagctttacctcctaggggaagcggagaaggaaggggatctttactgacttgtggcgacgtggaggagaaccccggccctatgtccgtgcctacccaagttctgggtttactgctgctctggctgacagacgctcgttgcgagatcgtgatgacccaatcccccgctacactctccgtgagccccggagagagggctactttaagctgtcgttcctcccagagcctcgtccacagaaacggcaatacctatttacactggtatttacagaagcccggtcagagccccaagttattaattcacaaagtcagcaacagattcagcggcgtgcccgatcgtttcagcggctccggcagcggaaccgactttacactcaagatcagcagagtggaggccgaggatctgggcgtgtacttctgctcccagtccacccatgtgccccctctcacattcggagccggaaccaagctggagctgaaaagaacagtcgccgccccctccgtcttcatttttcccccttccgatgagcagctgaagagcggcacagccagcgtggtctgtctgctcaacaatttttaccctcgtgaggccaaggtccagtggaaggtggataatgctctgcagtccggcaactcccaagaatccgtgacagagcaagactccaaggattccacctattctttatcctccaccctcactttaagcaaggctgactacgagaagcataaggtgtacgcttgtgaagtgacacaccaaggtttatccagccccgtgacaaagagcttcaataggggagagtgc

SEQ ID NO: 32 - protein, 444 aa, Artificial sequence
  1st polypeptide, GD2-14.18 DAR
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTPRKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 220 aa, Artificial sequence
  2nd polypeptide, GD2-14.18 DAR
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 722 aa, Artificial sequence
  Precursor  GD2-hu14.18 DAR
MEWSWVFLFFLSVTTGVHSEVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMSVPTQVLGLLLLWLTDARCDVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 2166 bp, Artificial sequence
  GD2-hu14.18 DAR precursor
atggaatggagctgggtctttctcttcttcctgtcagtaaccaccggtgtccactccgaagtgcaactcgttcaaagcggagccgaggtcgaaaaacccggagcctcagtcaagattagctgtaaagctagtggcagcagctttacagggtacaacatgaactgggtccgacagaacattgggaagtcactcgagtggattggggctattgacccgtactacggtggaacatcatacaatcagaaatttaaaggacgagcaaccctcacggtagacaaaagtacttccacggcgtatatgcatctcaagagtctcagaagtgaagacacagcggtctattattgtgtatctggtatggagtattggggccaaggtacgtccgtcactgtttcatctgctagcacaaaaggcccctccgtctttcctttagcccctagctccaaatccacctccggcggaacagccgccctcggatgtctcgtcaaagactacttccccgagcccgtgacagtgtcttggaattccggcgctttaacctccggagtgcacacctttcccgctgttttacagtccagcggactgtattctttaagctccgtcgtcaccgtgcctagcagctctttaggcacccagacctacatttgcaacgtcaaccacaagcctagcaacaccaaggtggacaaaagggtggagcctaagtcttgtgacaagacccacaccaagatcgaggtcatgtacccccccccctatttagacaacgagaaaagcaacggcacaattatccatgtgaaaggcaagcatctctgcccctcccctctgtttcccggaccctccaagcctttttgggtgctcgtggtggtgggcggcgtgctggcttgttattctttactggtgaccgtcgcctttatcatcttctgggtgaagaggggtcgtaagaagctgctgtacatcttcaagcagcccttcatgaggcccgttcaaaccacccaagaagaggacggctgcagctgtcgtttccccgaggaggaagagggaggatgcgagctgagggtgaagttctctcgtagcgccgatgcccccgcttatcagcaaggtcagaaccagttatacaacgagctgaatttaggtcgtagggaggagtacgacgtgctggacaagagaagaggtcgtgaccccgagatgggcggcaaacctcgtaggaagaacccccaagaaggtttatacaatgagctgcagaaggacaagatggccgaggcctacagcgagatcggcatgaagggcgagaggagaaggggcaagggccacgatggtttataccaaggtttaagcaccgccaccaaggacacctacgatgctttacacatgcaagctttacctcctcgtggaagcggcgaaggaaggggctctttactgacatgtggcgacgtggaggaaaaccccggccctatgtccgtgcccacacaagttctcggactgttattactgtggctgacagatgctcgttgcgatgtcgttatgacgcaaaccccgctttccttgccggttacgcctggagaaccagcttccatctcatgtcggtcttcacaatctctggttcatcgcaacggaaacacgtatcttcattggtatcttcagaagccaggccaatccccaaagctcctcattcacaaggtctctaacagattcagtggggttcctgacagattttccgggtctggatcaggtactgacttcacattgaaaattagccgcgtagaagctgaggacctgggcgtatacttttgttctcaatccacccatgtaccacctctcactttcggcgcaggaacgaaactggaactgaagagaaccgtggccgcccctagcgttttcattttcccccccagcgacgaacagctgaagtccggcacagcctccgtggtgtgtttactgaataacttctaccctcgtgaggctaaggtccaatggaaggtggataatgctttacagtccggaaattcccaagaaagcgtgaccgagcaagatagcaaagatagcacctacagcttaagctccacactgacactgtccaaagccgactacgagaaacacaaggtgtacgcttgtgaagtgacccatcaaggtttaagctcccccgtcacaaagagctttaacagaggagaatgc

SEQ ID NO: 36 - protein, 442 aa, Artificial sequence
  1st polypeptide, GD2-hu14.18 DAR
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 220 aa, Artificial sequence
  2nd polypeptide, GD2-hu14.18 DAR
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 15 aa, Artificial sequence
  G4S linker
GGGGSGGGGSGGGGS

SEQ ID NO: 39 - DNA, 195 bp, Artificial sequence
  JeT promoter
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcagttgaca

SEQ ID NO: 40 - DNA, 660 bp, Homo sapiens
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 41 - DNA, 650 bp, Homo sapiens
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 42 - DNA, 19 bp, Homo sapiens
cagggttctggatatctgt

SEQ ID NO: 43 - DNA, 20 bp, Artificial sequence
  Forward primer
  misc_feature (1)..(2): phosphorothioate linkage
  misc_feature (3)..(4): phosphorothioate linkage
  modified_base (3)..(3): cm
  misc_feature (4)..(5): phosphorothioate linkage
  modified_base (4)..(4): 2′-O-methyladenosine
  modified_base (5)..(5): cm
atcacgagcagctggtttct

SEQ ID NO: 44 - DNA, 24 bp, Artificial sequence
  Reverse primer
gacctcatgtctagcacagttttg

SEQ ID NO: 45 - DNA, 171 bp, Homo sapiens
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 46 - DNA, 161 bp, Homo sapiens
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 47 - protein, 10 aa, Artificial sequence
  IgG1 upper hinge sequence
EPKSCDKTHT

SEQ ID NO: 48 - protein, 4 aa, Artificial sequence
  IgG1 core hinge sequence
  MISC_FEATURE (3)..(3): X is P, R or S
CPXC

SEQ ID NO: 49 - protein, 9 aa, Artificial sequence
  lower hinge/CH2 sequence
PAPELLGGP

SEQ ID NO: 50 - protein, 12 aa, Artificial sequence
  Fc region (CH2)
SVFLFPPKPKDT

SEQ ID NO: 51 - protein, 23 aa, Artificial sequence
  hinge sequence including upper, core and lower hinge
EPKSCDKTHTCPPCPAPELLGGP

SEQ ID NO: 52 - protein, 9 aa, Artificial sequence
  family name for site-specific DNA endonucleases
LAGLIDADG

(19) United States
(12) Patent Application Publication
(10) Pub. No.: US 2024/0197881 A1
(43) Pub. Date: Jun. 20, 2024

(54) Dimeric Antigen Receptors (DARs) that Bind GD2

(71) Applicant: Sorrento Therapeutics, Inc., San Diego, CA (US)

(72) Inventors: Henry Hongjun Ji, Rancho Santa Fe, CA (US); Wenzhong Guo, San Diego, CA (US); Yanliang Zhang, San Diego, CA (US); Bei Bei Ding, San Diego, CA (US); Yang Wang, San Diego, CA (US); Gunnar F. Kaufmann, San Diego, CA (US)

(73) Assignee: Sorrento Therapeutics, Inc., San Diego, CA (US)

(21) Appl. No.: 18/287,255

(22) PCT Filed: Apr. 22, 2022
(86) PCT No.: PCT/US2022/026031

§ 371 Date: Oct. 17, 2023

Related U.S. Application Data
(60) Provisional application No. 63/188,284, filed on May 13, 2021.
(60) Provisional application No. 63/179,147, filed on Apr. 23, 2021.

(51) Int. Cl. A61K 39/00; A61P 35/00; C07K 14/705; C07K 14/725; C07K 16/30; C12N 5/0783

(52) U.S. Cl. CPC A61K 39/464471; A61K 39/4611; A61K 39/4631; A61P 35/00; C07K 14/7051; C07K 14/70517; C07K 14/70521; C07K 14/70578; C07K 16/3084; C12N 5/0636; C07K 2319/03; C12N 2510/00

(57) ABSTRACT

The present disclosure provides transgenic T cells expressing engineered dimeric antigen receptors (DARs) that bind GD2. where the DAR includes a heavy chain binding region on one polypeptide chain and a light chain binding region on a separate polypeptide chain. The two polypeptide chains that make up the dimeric antigen receptors can dimerize to form an antigen binding domain. The transgenic T cells can be used for directed cell therapy.

This application claims the benefit of priority under 35 U.S.C. 119 to U.S. provisional application No. 63/179,147, filed Apr. 23, 2021, and to U.S. provisional application No. 63/188,284, filed May 13, 2021, the entire contents of which are incorporated by reference in their entireties.

### TECHNICAL FIELD

The present disclosure provides dimeric antigen receptors (DAR) protein constructs that bind specifically to a target antigen, nucleic acids that encode the dimeric antigen receptors, vectors comprising the nucleic acids, and host cells harboring the vectors.

### BACKGROUND

GD2 is a disialoganglioside that is overexpressed on tumors of neuroectodermal origin, including human neuroblastoma and melanoma. In normal human tissues, GD2 expression is restricted to cerebellum and peripheral nerves. Murine anti-GD2 monoclonal antibody 14.18 was used to engineer a chimeric monoclonal antibody (ch14.18, referred to herein as 14.18) that includes the variable region of murine antibody 14.18 and constant regions of human IgG-K (Gillies, 1989 Journal of Immunological Methods 125:191-202). The humanized version of murine antibody 14.18 (hu14.18) includes variable region framework mutations designed to reduce immunogenicity of the antibody in humans (U.S. Pat. No. 7,169,904).

Adoptive immunotherapy by infusion of T cells engineered with chimeric antigen receptors (CARs) for redirected tumoricidal activity represents a potentially highly specific modality for the treatment of metastatic cancer. Different versions of CARs have been developed to target antigens associated with cancer. The first-generation CAR was engineered to contain a signaling domain (TCRC) that delivers an activation stimulus (signal 1) only (Geiger et al., J. Immunol. 162(10): 5931-5939, 1999; Haynes et al., J. Immunol. 166(1): 182-187, 2001) (Hombach et al. (Cancer Res. 61(5): 1976-1982, 2001; Hombach et al., J. Immunol. 167(11): 6123-6131, 2001; Maher et al., Nat. Biotechnol. 20(1): 70-75, 2002). T cells grafted with the first-generation CARs alone exhibited limited anti-tumor efficacy due to suboptimal activation (Beecham et al., J. Immunother. 23(6): 631-642, 2000). The second-generation CAR, immunoglobulin-CD28-T cell receptor (IgCD28TCR), incorporated a costimulatory CD28 (signal 2) into the first-generation receptor (Gerstmayer et al., J. Immunol. 158(10): 4584-4590, 1997; Emtage et al., Clin. Cancer Res. 14(24): 8112-8122, 2008; Lo, Ma et al., Clin. Cancer Res. 16(10): 2769-2780, 2010) resulting in CAR-T cells with a greater anti-tumor capacity (Finney et al., J. Immunol. 161(6): 2791-2797, 1998; Hombach et al., Cancer Res. 61(5): 1976-1982, 2001, Maher et al., Nat. Biotechnol. 20(1): 70-75, 2002). Various CAR variants have been developed by replacing the signal domains of TCRζ or CD28 with molecules with similar functions, such as FcRγ, 4-1BB and OX40 (Eshhar et al., Proc. Natl. Acad. Sci. USA 90(2): 720-724, 1993).

TCR CAR-T cells against various tumor antigens have been developed (Ma et al., Cancer Gene Ther. 11(4): 297-306, 2004; Ma et al., Prostate 61(1): 12-25, 2004; Lo et al., Clin. Cancer Res. 16(10): 2769-2780, 2010; Kong et al., Clin. Cancer Res. 18(21): 5949-5960, 2012; Ma et al., Prostate 74(3): 286-296, 2014; Katz et al., Clin. Cancer Res. 21(14): 3149-3159, 2015; Junghans et al., 2016 The Prostate, 76(14): 1257-1270). CAR-T cells targeting CD19, a molecule expressed on B cells, have shown success in treatment of B cell malignancies and have received FDA approval, with some trials showing a response rate of up to 70%. Nonetheless, CAR-T cells may show nonspecific activation, which may result in potentially serious adverse events through inappropriate immune activity.

### SUMMARY

Provided herein are engineered dimeric antigen receptors having first and second polypeptide chains that associate with each other to form an antigen binding domain that binds a GD2 molecule. The antigen binding domain is made up of an antibody heavy chain variable region located on the first polypeptide and an antibody light chain variable region located on the second polypeptide, or alternatively, an antibody light chain variable region located on the first polypeptide and an antibody heavy chain variable region located on the second polypeptide. The antibody heavy chain and light chain variable regions are followed by antibody constant regions (CH1 and CL, respectively), and the first polypeptide further includes a transmembrane domain and at least one intracellular signaling domain. When the first and second polypeptides of the DAR are expressed by a host cell, the first and second polypeptides associate with one another via disulfide bonds between antibody constant regions at the cell exterior allowing association of the heavy and light chain variable regions of the two polypeptides to form an antigen binding domain outside the cell. Assembly of the DAR via the antibody constant regions external to the cell thus generates a Fab fragment linked to the transmembrane and intracellular signaling domains of the first polypeptide. Host cells, such as T cells, expressing DARs can be used for cell-based therapy, for example in the treatment of cancer.

In a first aspect, provided herein is a dimeric antigen receptor (DAR) that binds GD2, where the DAR comprises: a) a first polypeptide comprising a plurality of polypeptide regions ordered from the amino terminus to the carboxyl terminus: (i) an antibody heavy chain variable region, (ii) an antibody heavy chain constant region (CH1), (iii) an optional hinge region, (iv) a transmembrane region, and (v) an intracellular region; and b) a second polypeptide comprising a plurality of polypeptide regions ordered from the amino terminus to the carboxyl terminus: (i) an antibody light chain variable region and (ii) an antibody light chain constant region (CL).

Alternatively, a DAR provided herein that binds GD2 can comprise: a first polypeptide chain comprising a plurality of polypeptide regions ordered from the amino terminus to the carboxyl terminus: (i) an antibody light chain variable region, (ii) an antibody light chain constant region (CL), (iii) an optional hinge region, (iv) a transmembrane region, and (v) an intracellular region; and b) a second polypeptide chain comprising a plurality of polypeptide regions ordered from the amino terminus to the carboxyl terminus: (i) an antibody heavy chain variable region and (ii) an antibody heavy chain constant region (CH1). The second polypeptide of a DAR as provided herein does not include a transmembrane region. The antibody heavy chain constant region and the antibody light chain constant region form a dimerization domain for association of the first and second polypeptides to form the DAR, and the antibody heavy chain variable region and the antibody light chain variable region form an antigen binding domain that binds GD2.

In some embodiments a GD2 DAR as provided herein comprises: a) a first polypeptide consisting essentially of: (i) an antibody heavy chain variable region, (ii) an antibody heavy chain constant region (CH1), (iii) an optional hinge region, (iv) a transmembrane region, and (v) an intracellular region; and b) a second polypeptide consisting essentially of: (i) an antibody light chain variable region and (ii) an antibody light chain constant region (CL). Alternatively a DAR provided herein that binds GD2 where the DAR can comprise: a) a first polypeptide consisting essentially of: (i) an antibody light chain variable region, (ii) an antibody light chain constant region (CL), (iii) an optional hinge region, (iv) a transmembrane region, and (v) an intracellular region; and b) a second polypeptide consisting essentially of: (i) an antibody heavy chain variable region and (ii) an antibody heavy chain constant region. The first and second polypeptides of the DAR dimerize via their antibody heavy chain constant region and the antibody light chain constant region that form one or more disulfide bonds. The two mature polypeptides, the transmembrane (first) polypeptide and the secreted (second) polypeptide, when produced by a host cell genetically modified to express genes encoding the first and second polypeptides, can assemble via cysteine bridges in their antibody constant domains at the cell exterior, forming a GD2 binding domain.

The antibody heavy chain variable region and antibody light chain variable region of a GD2 DAR polypeptide can be derived from a GD2 antibody, which can be, as nonlimiting examples, the 14.18 antibody or a humanized version (hu14.18). In some embodiments, the first polypeptide of a GD2 DAR includes the heavy chain variable region of 14.18 (SEQ ID NO:2) or a heavy chain variable region having at least 95%, at least 96%, at least 97%, at least 98%, or at least 99% identity to SEQ ID NO:2, and includes a light chain variable region of 14.18 (SEQ ID NO:5) or a light chain variable region having at least 95%, at least 96%, at least 97%, at least 98%, or at least 99% identity to SEQ ID NO:5. Alternatively, in some embodiments, the first polypeptide of a GD2 DAR includes the light chain variable region of 14.18 (SEQ ID NO:5) or a light chain variable region having at least 95%, at least 96%, at least 97%, at least 98%, or at least 99% identity thereto, and includes a heavy chain variable region of 14.18 (SEQ ID NO:2) or a heavy chain variable region having at least 95%, at least 96%, at least 97%, at least 98%, or at least 99% identity thereto. A heavy chain variable region having at least 95%, at least 96%, at least 97%, at least 98%, or at least 99% identity to SEQ ID NO:2 in various embodiments includes the heavy chain variable region CDR sequences of SEQ ID NO:2. A light chain variable region having at least 95%, at least 96%, at least 97%, at least 98%, or at least 99% identity to SEQ ID NO:5 in various embodiments includes the light chain variable region CDR sequences of SEQ ID NO:5.

In further embodiments, the first polypeptide of a GD2 DAR includes the heavy chain variable region of hu14.18 (SEQ ID NO:3) or a heavy chain variable region having at least 95%, at least 96%, at least 97%, at least 98%, or at least 99% identity to SEQ ID NO:3, and includes a light chain variable region of hu14.18 (SEQ ID NO:6) or a light chain variable region having at least 95%, at least 96%, at least 97%, at least 98%, or at least 99% identity to SEQ ID NO:6. Alternatively, in some embodiments, the first polypeptide of a GD2 DAR includes the light chain variable region of hu14.18 (SEQ ID NO:6) or a light chain variable region having at least 95%, at least 96%, at least 97%, at least 98%, or at least 99% identity thereto, and includes a heavy chain variable region of hu14.18 (SEQ ID NO:3) or a heavy chain variable region having at least 95%, at least 96%, at least 97%, at least 98%, or at least 99% identity to SEQ ID NO:3. A heavy chain variable region having at least 95%, at least 96%, at least 97%, at least 98%, or at least 99% identity to SEQ ID NO:3 in various embodiments includes the heavy chain variable region CDR sequences of SEQ ID NO:3. A light chain variable region having at least 95%, at least 96%, at least 97%, at least 98%, or at least 99% identity to SEQ ID NO:6 in various embodiments includes the light chain variable region CDR sequences of SEQ ID NO:6.

The heavy chain variable region of a first or second DAR polypeptide is followed by a heavy chain constant region (CH1), e.g., SEQ ID NO:4 or a constant region having at least 95%, at least 96%, at least 97%, at least 98%, or at least 99% identity to SEQ ID NO:4. The light chain variable region of a first or second DAR polypeptide is followed by a light chain constant region, which may be a light chain kappa constant region (SEQ ID NO:7, or a constant region having at least 95%, at least 96%, at least 97%, at least 98%, or at least 99% identity thereto) or a light chain lambda constant region (SEQ ID NO:8, or a constant region having at least 95%, at least 96%, at least 97%, at least 98%, or at least 99% identity thereto).

The hinge region of the first polypeptide is optional, and when present can comprise a hinge sequence from an antibody or other immunological molecule or a portion thereof, for example, can be selected from a hinge region of an IgG, IgA, IgM, IgE or IgD or a portion of any thereof. A hinge region, including but not limited to at least a portion of a hinge region derived from one or more naturally-occurring proteins comprise three, four, five, six, seven, eight, nine, or ten or more amino acids, for example, between about three and about twenty amino acids, between about ten and about thirty amino acids, between about twenty and about fifty amino acids, between about thirty and about sixty amino acids, between about forty and about eighty amino acids, between about fifty and about one hundred amino acids, between about sixty and about 120 amino acids, or between about eighty and about 150 amino acids or longer. In further nonlimiting examples, the hinge region of the first polypeptide of the DAR can comprise a CD8α hinge region (SEQ ID NO: 10), a CD28 hinge region (SEQ ID NO:9), or a CD8 α/CD28 hinge region (SEQ ID NO:11), or can comprise a hinge region having at least 95%, at least 96%, at least 97%, at least 98%, or at least 99% identity to any thereof.

The transmembrane region of the first polypeptide can comprise a transmembrane region of CD8 (SEQ ID NO:13), CD28 (SEQ ID NO:12), 4-1BB (SEQ ID NO:14), or CD3ζ (SEQ ID NO:15), as nonlimiting examples, or a transmembrane region having at least 95%, at least 96%, at least 97%, at least 98%, or at least 99% identity to any thereof.

The intracellular region of the first polypeptide can comprise one or more intracellular amino acid sequences selected from a group consisting of 4-1BB intracellular region (SEQ ID NO:16), CD3ζ having ITAMs 1, 2 and 3 (SEQ ID NO:19), CD3ζ having ITAM 1 (SEQ ID NO:20), CD3ζ having ITAM 3 (SEQ ID NO:22), or an intracellular region of any of CD28 (SEQ ID NO:17), CD27, OX40 (SEQ ID NO:18), CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2 and/or CD226, or an intracellular amino acid sequence having at least 95% identity to any thereof. In some embodiments, the intracellular region comprises a CD3ζ intracellular region having ITAMs 1, 2 and 3 (SEQ ID NO:19) or an intracellular region having least 95%%, at least 96%, at least 97%, at least 98%, or at least 99% identity to SEQ ID NO: 19. In some embodiments, the intracellular region comprises a 4-1BB intracellular region (SEQ ID NO:16) or an intracellular region having least 95%%, at least 96%, at least 97%, at least 98%, or at least 99% identity to SEQ ID NO: 16. In some embodiments the intracellular domain includes or consists essentially of the 4-1BB intracellular region (SEQ ID NO:16) and the CD33 intracellular region having ITAMs 1, 2 and 3 (SEQ ID NO:19).

In some embodiments of a GD2 DAR, the first polypeptide comprises the amino acid sequence of SEQ ID NO:32 or an amino acid sequence having at least 95%%, at least 96%, at least 97%, at least 98%, or at least 99% identity thereto, and the second polypeptide comprises the amino acid sequence of SEQ ID NO:33 or an amino acid sequence having at least 95%, at least 96%, at least 97%, at least 98%, or at least 99% identity thereto. In some embodiments of a GD2 DAR, the first polypeptide comprises the amino acid sequence of SEQ ID NO:36 or an amino acid sequence having at least 95%%, at least 96%, at least 97%, at least 98%, or at least 99% identity thereto, and the second polypeptide comprises the amino acid sequence of SEQ ID NO:37 or an amino acid sequence having at least 95%, at least 96%, at least 97%, at least 98%, or at least 99% identity thereto.

In a further aspect, nucleic acid molecules are provided that encode a GD2 DAR, such as any described herein. For example, one or more nucleic acid molecules can encode one or more precursor polypeptides that can be expressed by a host cell to produce a DAR having a first and second polypeptide as described herein. In some embodiments one or more nucleic acid molecules that encode the DAR encode one or more precursor polypeptides that may include, for example one or more leader sequences (signal peptides) for membrane insertion or secretion of a produced first or second polypeptide. Further a nucleic acid molecule that encodes the first and second polypeptide of a DAR may include a peptide “self-cleavage” or “2A” sequence between sequences encoding the first and second such that the nucleic acid molecule encodes a single open reading frame that includes sequences of both the mature first polypeptide and the mature second polypeptide, where both polypeptide-encoding sequences are preceded by a sequence encoding a signal peptide (i.e., are encoded as precursor polypeptides), and the first and second polypeptide-encoding sequences are separated by a sequence encoding a 2A peptide (e.g., SEQ ID NO:26, SEQ ID NO:27, SEQ ID NO:28, or SEQ ID NO:29) or other sequence that results in the production of two separate polypeptides. As nonlimiting examples, a nucleic acid molecule as provided herein that encodes a DAR can encode a precursor polypeptide of SEQ ID NO:30, or a precursor polypeptide having at 95%, at least 96%, at least 97%, at least 98%, or at least 99% identity to SEQ ID NO:30, where a host cell expressing the DAR-encoding sequence produces the first and second polypeptides, or can encode a precursor polypeptide of SEQ ID NO:34, or a precursor polypeptide having at 95%, at least 96%, at least 97%, at least 98%, or at least 99% identity to SEQ ID NO:34, where a host cell expressing the DAR-encoding sequence produces the first and second polypeptides.

Other configurations of nucleic acid molecules that can encode a DAR as provided herein include, without limitation, open reading frames encoding first and second precursor polypeptides of the DAR where the open reading frames are separated by an internal ribosome entry site (IRES) that can allow for the translation of two polypeptides from the same promoter. Also considered is a nucleic acid molecule encoding a first polypeptide and a second polypeptide as separate open reading frames each with its own promoter. Further considered are two vectors that together encode both polypeptides of the DAR. In these embodiments, signal peptides will typically be included at the N-terminus of each polypeptide to ensure membrane integration of the first polypeptide and secretion of the second polypeptide. Nonlimiting examples of signal peptides are provided as SEQ ID NOs:23, 24, and 25.

A nucleic acid molecule encoding a DAR precursor polypeptide can include a promoter operably linked to the one or more sequences encoding the first and second polypeptides of the DAR. Provided herein are expression cassettes that include a promoter operably linked to a nucleic acid sequence encoding a DAR precursor polypeptide, where the DAR precursor polypeptide may encode one or both polypeptides of the DAR. A promoter can be, for example, a promoter active in eukaryotic cells, for example, a promoter active in mammalian cells. As nonlimiting examples, the promoter can be a JeT promoter (SEQ ID NO:39), a CMV promoter, an HTLV promoter, an EF1α promoter, or an EF1α/HTLV hybrid promoter.

In some embodiments provided herein are nucleic acid expression cassettes that encode at least one polypeptide of a GD2 DAR, where the expression cassette is flanked by sequences of a human gene locus, for example, sequences of the human TRAC gene.

In some exemplary embodiments a nucleic acid as provided herein can encode a precursor polypeptide of SEQ ID NO:30 that encodes a first and second polypeptide of a GD2 DAR and can have at least 65%, at least 70%, at least 75%, at least 80%, or at least 85%, at least 90%, or at least 95% identity to SEQ ID NO:31. In some exemplary embodiments a nucleic acid as provided herein can encode a precursor polypeptide of SEQ ID NO:34 that encodes a first and second polypeptide of a GD2 DAR and can have at least 65%, at least 70%, at least 75%, at least 80%, or at least 85%, at least 90%, or at least 95% identity to SEQ ID NO:35.

Also provided herein is the use of a nucleic acid molecule encoding a GD2 DAR, such as any disclosed herein, for the manufacture of a medicament for treating cancer. The medicament can be, for example, genetically engineered cells, such as T cells or NK cells, expressing the GD2 DAR.

In another aspect, genetically engineered host cells are provided that include one or more nucleic acid molecules as provided herein for expressing a GD2 DAR such as any disclosed herein. The cells can be, as nonlimiting examples, T cells or NK cells. In some embodiments, one or more nucleic acid molecules encoding at least one DAR polypeptide is integrated into the genome of the engineered host cell. For example, one or more nucleic acid molecules encoding at least one DAR polypeptide can integrated into the TRAC locus of the engineered host cell. In various embodiments provided herein, a genetically engineered host cell expresses a GD2 DAR as provided herein and does not express the T cell receptor. Further provided herein is a population of T cells in which at least 20%, at least 30%, at least 40%, or at least 50% of the cells express a GD2 DAR and do not express a T cell receptor. Further provided herein is a population of T cells in which at least 90% or at least 95% of the cells that express a GD2 DAR do not express a T cell receptor.

In some embodiments the genetically engineered cells that express a GD2 DAR are T cells (DAR-T cells), e.g., human T cells. In some embodiments the DAR-T cells are primary T cells. In alternative embodiments the genetically engineered cells that express a GD2 DAR are Natural Killer (NK) cells (DAR-NK cells), e.g., human NK cells. In some embodiments the DAR-NK cells are primary NK cells

In a further aspect provided herein are methods of treating cancer by administering anti-GD2 DAR-T cells to a subject having cancer. The cells can be administered in a single dose or multiple doses, for example of from about 10⁵ to about 10⁹ cells. The cells can be cells of a population where at least 10%, at least 20%, at least 30%, at least 40%, or at least 50% of the cells express the DAR construct and less than 10%, less than 5%, or less than 1% of the cells express the endogenous T cell receptor (e.g., less than 1% of the cells are CD3-positive).

Further provided are genetically engineered cells that express a GD2 DAR as disclosed herein and/or that include a nucleic acid molecule encoding a GD2 DAR, such as any described herein, for use in a method of treating cancer by administering the genetically engineered GD2 DAR-expressing cells a subject having cancer.

Additionally provided is the use of genetically engineered cells as provided herein that express a GD2 DAR such as any disclosed herein for the manufacture of a medicament for treating cancer.

Further embodiments according to this disclosure are set forth in the claims and the detailed description.

### DESCRIPTIONS OF THE DRAWINGS

FIG. 1A-D provides diagrams showing the organization and domains of four exemplary configurations of dimeric antigen receptors. A) is a diagram of a DAR showing the disulfide linkage between the constant antibody regions of the first and second polypeptides, where the first polypeptide that includes a transmembrane domain and two intracellular signaling regions includes a heavy chain variable region of an antibody and the second polypeptide that does not include a transmembrane domain or any intracellular domains includes a light chain variable region of an antibody. B) is a diagram of a DAR showing the disulfide linkage between the constant antibody regions of the first and second polypeptides, where the first polypeptide that includes a transmembrane domain and three intracellular signaling regions includes a heavy chain variable region of an antibody and the second polypeptide that does not include a transmembrane domain or any intracellular domains includes a light chain variable region of an antibody. C) is a diagram of a DAR showing the disulfide linkage between the constant antibody regions of the first and second polypeptides, where the first polypeptide that includes a transmembrane domain and two intracellular signaling regions includes a light chain variable region of an antibody and the second polypeptide that does not include a transmembrane domain or any intracellular domains includes a heavy chain variable region of an antibody. D) is a diagram of a DAR showing the disulfide linkage between the constant antibody regions of the first and second polypeptides, where the first polypeptide that includes a transmembrane domain and three intracellular signaling regions includes a light chain variable region of an antibody and the second polypeptide that does not include a transmembrane domain or any intracellular domains includes a heavy chain variable region of an antibody.

FIG. 2A-D provides diagrams showing the organization and domains of four exemplary configurations of precursors of dimeric antigen receptors that in some embodiments may be encoded by nucleic acid molecules that can be transfected into host cells for expression of DARs. A and B) provide diagrams of precursor polypeptides for the expression of the DARs shown in FIG. 1B and FIG. 1A, respectively, where the precursor polypeptide includes a leader sequence to direct the first polypeptide to the membrane, a “self cleaving sequence” to allow the precursor polypeptides to be expressed as a first and second polypeptide, and a second leader sequence for secretion of the second polypeptide. C) and D) provide diagrams of precursor polypeptides for the expression of the DARs shown in FIG. 1D and FIG. 1C, respectively, where the precursor polypeptide includes a leader sequence to direct the first polypeptide to the membrane, a “self cleaving sequence” to allow the precursor polypeptides to be expressed as a first and second polypeptide, and a second leader sequence for secretion of the second polypeptide.

FIG. 3 shows the results of flow cytometry of several cell lines including NCI-H524, SK-MEL-5, K562 and H460 using a labeled antibody to GD2 (x-axis).

FIG. 4 shows the results of flow cytometry detecting expression of GD2(14.18) CAR (anti-GD2(14.18)-scFv-28z), GD2(hu14.18) CAR (anti-GD2(hu14.18)-scFv-28z), or

GD2(14.18) DAR (anti-GD2(14.18)-DAR-BBz) in transgenic T cells (x axis) fourteen days after transfection. Very few transgenic CAR-T and DAR-T expressing transgenic cells express CD3 (y axis).

FIG. 5 shows the results of flow cytometry monitoring of expansion of transgenic T cells expressing either GD2 CAR or GD2-DAR over time in co-culture with cell lines that either express GD2 (GD2+) or have very low to no expression of GD2 (indicated as GD2−). The column labeled “T Only” designates CAR T cells or DAR T cells cultured alone. AntiGD2(14.18)-scFv-28z designates the CAR construct made with the scFv of the 14.18 (chimeric) anti-GD2 antibody; antiGD2(hu14.18)-scFv-28z designates the CAR construct made with the scFv of the hu14.18 (humanized) anti-GD2 antibody; antiGD2(14.18)DAR-BBz designates the DAR construct made with the heavy chain and light chain sequences of the 14.18 (chimeric) anti-GD2 antibody. GD2 construct expression along the x-axis; CD3 expression along the y-axis.

FIG. 6A is a graph showing the percent in vitro cytotoxicity using transgenic T cells expressing either GD2 CAR or GD2 DAR as effectors and SK-MEL-5 (GD2+) target cells. Populations used in the assays that were generated from isolated T cells transfected with CAR or DAR constructs are designated with the letter ‘T’; populations used in the assays that were generated from PBMCs transfected with CAR or DAR constructs are designated with the letter ‘P’. AntiGD2(14.18)-scFv-28z designates the CAR construct made with the scFv of the 14.18 (chimeric) anti-GD2 antibody; antiGD2(hu14.18)-scFv-28z designates the CAR construct made with the scFv of the hu14.18 (humanized) anti-GD2 antibody; antiGD2(14.18)DAR-BBz designates the DAR construct made with the heavy chain and light chain sequences of the 14.18 (chimeric) anti-GD2 antibody. TRAC KO are control T cells knocked out for the T cell receptor but not transfected with a CAR or DAR construct. Also indicated in the legend is the percentage of the population of transfected T cells or PBMCs used in the assays that expressed the GD2 CAR or DAR construct. FIG. 6B is a graph showing the percent in vitro cytotoxicity using transgenic T cells expressing either GD2 CAR or GD2-DAR as effectors and NCI-H460 (GD2-) target cells. Cell killing was measured using the xCELLigence® Real-Time Cell Analysis (RTCA) system (Agilent). Cell populations as in FIG. 6A.

FIG. 7A is a bar graph showing the level of IFN-gamma release (48 hours post-target stimulation) from either T cells having TRAC knock-out only (TRAC KO), or transgenic T cells expressing either GD2 CAR or GD2-DAR constructs. The target cells include T cells only (no target control), NCI-H524 (GD2+) cells, or K562 (GD2-) cells. Each assay set includes (from left to right): TRAC KO; GD2(14.18) CAR (isolated T cells); GD2(hu14.18) CAR (isolated T cells); GD2(14.18) DAR (isolated T cells); GD2(14.18) CAR (PBMCs); GD2(hu14.18) CAR (PBMCs); GD2(14.18) DAR (PBMCs). Arrows indicate the values for GD2(14.18) DAR populations.

FIG. 7B is a bar graph showing the level of GM-CSF release (48 hours post-target stimulation) from either T cells having TRAC knock-out only (TRAC KO), or transgenic T cells expressing either GD2 CAR or GD2-DAR constructs. The assays include a no target control (T cells only), or as target cells NCI-H524 (GD2+) or K562 (GD2-) cells. Each assay set includes (from left to right): TRAC KO; GD2(14.18) CAR (isolated T cell); GD2(hu14.18) CAR (isolated T cell); GD2(14.18) DAR (isolated T cell); 14.18 CAR (PBMC); hu14.18 CAR (PBMC); 14.18 DAR (PBMC).

FIG. 8 provides the in vivo images up to 7 weeks after treatment of mice inoculated with SK-MEL-5 tumor cells and then treated with PBS only, TRAC knockout T cells, GD2(hu14.18) DAR-T cells where the DAR included the hu14.18 heavy and light chain variable regions (labeled as anti-GD2 hDAR T), and GD2(14.18) DAR-T cells where the DAR included the 14.18 heavy and light chain variable regions (labeled as anti-GD2 mDAR T).

FIG. 9A is a graph of average tumor volume over the time of treatment of the groups of mice shown in FIG. 8. FIG. 9B is a graph of average body weight over the time of treatment of the groups of mice shown in FIG. 8.

FIG. 10 provides survivorship curves of the mice shown in FIG. 8.

### DESCRIPTION

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The headings provided herein are solely for the convenience of the reader, and are not limitations of the various aspects of the disclosure, which aspects can be understood by reference to the specification as a whole.

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N. Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also encompassed. The phrase “consisting essentially of” indicates that the specified materials or steps are included as well as those that do not materially affect the basic and novel characteristic(s) of the composition or method described.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., +10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide”, “polypeptide chain” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules and mature molecule. Precursor molecules include those that have not yet been subjected to cleavage, for example cleavage by a secretory signal peptide or by non-enzymatic cleavage at certain amino acid residue. Polypeptides in include mature molecules that have undergone cleavage. These terms encompass native proteins, recombinant proteins and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 2-6 or more polypeptide chains) can associate with each other, via covalent and/or non-covalent association, to form a polypeptide complex. Association of the polypeptide chains can also include peptide folding. Thus, a polypeptide complex can be dimeric, trimeric, tetrameric, or higher order complexes depending on the number of polypeptide chains that form the complex. Dimeric antigen receptors (DAR) comprising two polypeptide chains are described herein.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof. In one embodiment, nucleic acids comprise one type of polynucleotide or a mixture of two or more different types of polynucleotides. Nucleic acids encoding dimeric antigen receptors (DAR) or antigen-binding portions thereof, are described herein.

The term “recover” or “recovery” or “recovering”, and other related terms, refers to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide (leader peptide sequence) sequence which mediates secretion of the expressed protein from a host cell (e.g., from a mammalian host cell). The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiment to protein or polynucleotides that are substantially free of other molecules of the same species, for example other protein or polynucleotides having different amino acid or nucleotide sequences, respectively. The purity or homogeneity of the desired molecule can be assayed using techniques well known in the art, including low resolution methods such as gel electrophoresis and high resolution methods such as HPLC or mass spectrometry. In one embodiment, isolated precursor polypeptides, and first and second polypeptide chains, of the DAR or antigen-binding portions thereof, of the present disclosure are isolated.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “leader sequence” or “leader peptide” or “peptide signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length. A leader sequence can direct transport of a precursor polypeptide from the cytosol to the endoplasmic reticulum. In one embodiment, a leader sequence includes signal sequences comprising CD8α, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence MEWSWVFLFFLSVTTGVHS (SEQ ID NO. 23).

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins comprising dimeric antigen receptors (DAR) are described herein.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having DARstructures with immunoglobulin-like properties that bind specifically to a target antigen (e.g., GD2 antigen) are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (“Kabat numbering”). Other numbering systems for the amino acids in immunoglobulin chains include IMGT.RTM. (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Antibodies-like molecules comprising dimeric antigen receptors (DAR) are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Dimeric antigen receptors (DAR) having antibody heavy chain variable regions and antibody light chain variable regions that form antigen binding domains are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ M or less. In one embodiment, dimeric antigen receptors (DAR) that bind specifically to their target antigen (e.g., GD2 antigen) are described herein.

In one embodiment, binding specificity of an antibody or antigen binding protein or antibody fragment can be measure by ELISA, radioimmune assay (RIA), electrochemiluminescence assays (ECL), immunoradiometric assay (IRMA), or enzyme immune assay (EIA).

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. In one embodiment, dimeric antigen receptors (DAR) or antigen-binding portions thereof that bind an epitope of GD2 antigen are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. In one embodiment, dimeric antigen receptors comprising a Fab fragment joined to a hinge, transmembrane and intracellular signaling regions are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or VL domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). In one embodiment, dimeric antigen receptors comprising a Fab fragment joined to a hinge, transmembrane and intracellular signaling regions are described herein.

A single-chain antibody (scFv) is an antibody in which a V_{L} and a V_{H} region are joined via a linker (e.g., a synthetic sequence of amino acid residues) to form a continuous protein chain. In one embodiment the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the dimeric antigen receptors (DAR) described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Dimeric antigen receptors (DAR) comprising fully human antibody heavy chain variable region and fully human antibody light chain variable regions are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. For example, certain amino acids in the framework and constant domains of the heavy and/or light chains of the non-human species antibody may be mutated to produce a humanized antibody. In other examples, the constant domain(s) from a human antibody may be fused to the variable domain(s) of a non-human species. In additional embodiments the CDR sequences of the non-human antibody may be substituted for CDRs in a human antibody or otherwise engineered into the framework of a human antibody. In other examples, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a non-human antibody. In another embodiment, all of the CDRs are derived from a human antibody. In another embodiment, the CDRs from more than one non-human antibody are mixed and matched in a chimeric antibody. For instance, a chimeric antibody may comprise a CDR1 from the light chain of a first non-human antibody, a CDR2 and a CDR3 from the light chain of a second non-human antibody, and the CDRs from the heavy chain from a third antibody. The framework sequences outside of the CDR sequences may be human antibody sequences, for example. In other examples, the CDRs can originate from different species such as human and mouse, or human and rabbit, or human and goat. One skilled in the art will appreciate that other combinations are possible. In some embodiments, including some embodiments disclosed herein, a chimeric GD2 antibody, such as the chimeric 14.18 antibody, includes a variable domains of a GD2 antibody of a nonhuman species (e.g., a mouse) fused to the constant domains of a human antibody. For example, a chimeric GD2 heavy chain or portion thereof used in a recombinant receptor as disclosed herein can include the heavy chain variable region of the mouse 14.18 antibody fused to a human antibody constant region, and a chimeric GD2 light chain or portion thereof used in a recombinant receptor as disclosed herein can include the light chain variable region of the mouse 14.18 antibody fused to a human antibody constant region.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising full-length heavy chains and full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In one embodiment, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region can be derived from is a hinge region of a naturally-occurring protein, such as a CD8 hinge region or a fragment thereof, a CD8α hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In one embodiment, the hinge region comprises any one or any combination of two or more regions comprising an upper, core or lower hinge sequences from an IgG1, IgG2, IgG3 or IgG4 immunoglobulin molecule. In one embodiment, the hinge region comprises an IgGI upper hinge sequence EPKSCDKTHT (SEQ ID NO: 47). In one embodiment, the hinge region comprises an IgG1 core hinge sequence CPXC, wherein X is P, R or S (SEQ ID NO: 48). In one embodiment, the hinge region comprises a lower hinge/CH2 sequence PAPELLGGP (SEQ ID NO: 49). In one embodiment, the hinge is joined to an Fc region (CH2) having the amino acid sequence SVFLFPPKPKDT (SEQ ID NO: 50). In one embodiment, the hinge region includes the amino acid sequence of an upper, core and lower hinge and comprises EPKSCDKTHTCPPCPAP ELLGGP (SEQ ID NO: 51). In one embodiment, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions, and may or may not include a portion of the hinge region. In one embodiment, two polypeptide chains each carrying a half Fc region can dimerize to form an Fc region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. In one embodiment, the Fc region can include a mutation that increases or decreases any one or any combination of these functions. An Fc region can bind an Fc receptor, including FcγRI (e.g., CD64), FcγRII (e.g., CD32) and/or FcγRIII (e.g., CD16a). An Fc region can bind a complement component Clq. In one embodiment, the Fc domain comprises a LALA-PG mutation (e.g., equivalent to L234A, L235A, P329G) which reduces effector function. In one embodiment, the Fc domain mediates serum half-life of the protein complex, and a mutation in the Fc domain can increase or decrease the serum half-life of the protein complex. In one embodiment, the Fc domain affects thermal stability of the protein complex, and mutation in the Fc domain can increase or decrease the thermal stability of the protein complex.

The term “labeled antibody” or related terms as used herein refers to antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens).

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of an antibody may be similar but not identical to any of the amino acid sequences of the antigen-binding portions and/or antibody constant region of a DAR described herein. The similarities between the antibody and the polypeptides can be at least 95%, or at or at least 96% identical, or at least 97% identical, or at least 98% identical, or at least 99% identical, to a sequence of any of the polypeptides that make up the DAR or antigen-derived portions thereof that are described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The term “Chimeric Antigen Receptor” or “CAR” refers to a single chain fusion protein comprising an extracellular antigen-binding protein that is fused to an intracellular signaling domain. The CAR extracellular binding domain is a single chain variable fragment (scFv or sFv) derived from fusing the variable heavy and light regions of a monoclonal antibody, such as a human monoclonal antibody. In one embodiment, a CAR comprises (i) an antigen binding protein comprising a heavy chain variable (VH) domain and a light chain variable (VL) domain wherein the VH and VL domains are joined together by a peptide linker; (ii) a hinge domain, (iii) a transmembrane domain; and (iv) an intracellular domain comprising an intracellular signaling sequence. The disclosed constructs are DARs which are distinct from CARs in that DARs do not use a single chain antibody for targeting but instead use separate heavy and light chain variable domain regions.

As used herein the term “dimeric antigen receptor” (DAR) refers to an engineered receptor comprising two polypeptide chains: a first polypeptide comprising an antigen binding region of an antibody (a heavy chain variable region or light chain variable region), followed by an antibody constant region, a transmembrane region, and an intracellular signaling region, and a second polypeptide comprising an antigen binding region (a light chain variable region or heavy chain variable region) followed by an antibody constant region. Where the first polypeptide includes a heavy chain variable region, the second polypeptide includes a light chain variable region, and vice versa, such that assembly of the DAR via the antibody constant regions external to the cell generates a Fab fragment linked to transmembrane and intracellular signaling domains of the first polypeptide. The first polypeptide is anchored to the cell membrane via the transmembrane domain, whereas the second polypeptide lacks a transmembrane domain and is secreted to the cell exterior where it assembles with the second polypeptide. The two polypeptide chains that make up the dimeric antigen receptors can dimerize to form a protein complex and have antibody-like properties as they bind specifically to a target antigen. The dimeric antigen receptors can be used for directed cell therapy.

The present disclosure provides transgenic T cells engineered to express anti-GD2 constructs having an antigen-binding extracellular portion, optional hinge portion, transmembrane portion, and an intracellular portion having co-stimulatory and/or intracellular signaling regions. a Fab fragment joined to a transmembrane region and intracellular regions. In one embodiment, the DAR construct includes an optional hinge region between the Fab fragment and the transmembrane region. In some embodiments, the presently disclosed DAR structures provide unexpected and surprising results, e.g., based on comparing a DAR structure having a Fab format antibody to a CAR structure having an scFv format of the same antibody. Moreover, the DAR and CAR formats can be directly compared because the hinge regions, transmembrane regions and two intracellular regions can be the same. Yet the DAR format can provide superior results relative to the corresponding CAR format in binding to cells expressing the target antigen, antigen-induced cytokine release and/or antigen-induced cytotoxicity.

The present disclosure provides DAR constructs comprising a heavy chain binding region on one polypeptide chain and a light chain binding region on a separate polypeptide chain. The two polypeptide chains that make up the dimeric antigen receptors can dimerize to form a protein complex. The dimeric antigen receptors have antibody-like properties as they bind specifically to a target antigen. The dimeric antigen receptors can be used for directed cell therapy.

Dimeric antigen receptors (DARs) and their various configurations and domains, constructs encoding DARs, and cells expressing DARs and their uses in cell therapy are also disclosed in WO 2019/173837 and WO 2021/046445, both of which are incorporated by reference herein in their entireties.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the dimeric antigen receptors (DAR) or antigen-binding portions thereof that are described herein.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been introduced with exogenous nucleic acid (transgene). The host cell includes the primary subject cell and its progeny. Exogenous nucleic acids encoding at least a portion of any of the dimeric antigen receptors (DARs) or antigen-binding portions thereof that are described herein can be introduced into a host cell. Expression vectors comprising at least a portion of any of the dimeric antigen receptors (DARs) or antigen-binding portions thereof that are described herein can be introduced into a host cell, and the host cell can express polypeptides comprising at least a portion of the DAR or antigen-binding portions thereof that are described herein.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. The host cell (or a population thereof) includes immortalized cell lines. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome, or can harbor an extrachromosomal expression vector. In one embodiment, host cells and populations thereof can harbor an extrachromosomal vector that is present after several cell divisions or is present transiently and is lost after several cell divisions.

Transgenic host cells can be prepared using non-viral methods, including well-known designer nucleases including zinc finger nucleases, TALENS, meganucleases, or by gene editing using CRISPR/Cas. A transgene can be introduced into a host cell's genome using a zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a CAR or DAR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA. Transgenic host cells can be prepared using a meganuclease which acts as a site-specific, rare-cutting endonuclease that recognizes a recognition site on double-stranded DNA about 12-40 base pairs in length. Meganucleases include those from the LAGLIDADG (SEQ ID NO: 52) family found most often in mitochondria and chloroplasts of eukaryotic unicellular organisms. An example of a Meganuclease system used to modify genomes is described for example in U.S. Pat. No. 9,889,160. Transgenic host cells can be prepared using CRISPR (Clustered Regularly Interspaced Short Palindromic Repeats).

CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Nonlimiting examples of Cas endonucleases that may be employed include Cas9, Cas12a, CasX, and related enzymes including variants of Cas9, Cas12a, and CasX and enzymes such as MAD7 and variants thereof, CeCpf1, , PrCpf1, Lb2Cpf1 and Lb2Cpf1-KY, etc. Examples of CRISPR/Cas systems used to modify genomes are described for example in U.S. Pat. Nos. 8,697,359; 10,000,772; 9,790,490; and 10,570,415 and U.S. Patent Application Publication Nos. US 2020/0109382, US 2021/0309981, and US 2021/0155911, all of which are incorporated by reference herein in their entireties. The use of CRISPR RNA-guided endonucleases can also be used to disrupt genes whose expression may be dispensible or undesirable and in some embodiments insertion of a CAR or DAR construct (such as a GD2 DAR construct) can be targeted to a gene encoding a TCR chain, e.g., a TRAC or TRBC gene, such that the TCR is not expressed in cells that express the CAR or DAR. CRISPR/Cas systems and methods for construct integration, gene knockout, and simultaneous knockin/knockout at a targeted locus are described, for example, in US 2020/0224160; WO 2020/176740, and WO 2020/185867, all of which are incorporated by reference herein in their entireties.

In various embodiments, transgenic host cells can be prepared using a zinc finger nuclease, TALEN or CRISPR/Cas system, and the host target site can be a TRAC gene (T Cell Receptor Alpha Constant). The donor DNA can include for example any of the nucleic acids encoding a CAR or DAR construct described herein. Electroporation, nucleofection or lipofection can be used to co-deliver into the host cell the donor DNA with the zinc finger nuclease, TALEN or CRISPR/Cas system.

Transgenic host cells can also be prepared by transducing host cells (e.g., T cells) with a retroviral vector carrying a nucleic acid encoding a CAR or DAR construct. The transduction can be performed essentially as described in Ma et al., 2004 The Prostate 61:12-25; and Ma et al., The Prostate 74(3):286-296, 2014 (the disclosures of which are incorporated by reference herein in their entireties). The retroviral vector can be transfected into a Phoenix-Eco cell line (ATCC) using FuGene reagent (Promega, Madison, WI) to produce ecotropic retrovirus, then harvest transient viral supernatant (Ecotropic virus) can be used to transduce PG13 packaging cells with Gal-V envelope to produce retrovirus to infect human cells. Viral supernatant from the PG13 cells can be used to transduce activated T cells (or PBMCs) two to three days after CD3 or CD3/CD28 activation. Activated human T cells can be prepared by activating normal healthy donor peripheral blood mononuclear cells (PBMC) with 100 ng/ml mouse anti-human CD3 antibody OKT3 (Orth Biotech, Rartian, NJ) or anti-CD3, anti-CD28 TransAct (Miltenyi Biotech, German) as manufacturer's manual and 300-1000 U/ml IL2 in AIM-V growth medium (GIBCO-Thermo Fisher scientific, Waltham, MA) supplemented with 5% FBS for two days. Approximately 5×10⁶ activated human T cells can be transduced in a 10 ug/ml retronectin (Takara Bio USA) pre-coated 6-well plate with 3 ml viral supernatant and centrifuged at 1000 g for about 1 hour at approximately 32° C. After transduction, the transduced T cells can be expanded in AIM-V growth medium supplemented with 5% FBS and 300-1000 U/ml IL2.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NSO, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CVI/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed or transfected) with an exogenous nucleic acid either to be expressed or not to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more polypeptides that comprise a DAR or antigen-binding portions thereof are described herein.

The host cell or the population of host cells comprise T lymphocytes (e.g., T cells, regulatory T cells, gamma-delta T cells, and/or cytotoxic T cells), NK (natural killer) cells, macrophages, dendritic cells, mast cells, eosinophils, B lymphocytes, monocytes. In some embodiments the host cells are T cells and the cells expressing the GD2 DAR may be referred to as GD2 DAR-T cells. In some embodiments, the host cells are NK cells and the cells expressing the GD2 DAR may be referred to as GD2 DAR-NK cells. In some embodiments, the NK cells comprise cord blood-derived NK cells and/or placenta-derived NK cells.

Polypeptides of the present disclosure (e.g., dimeric antigen receptors (DARs)) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions permissive for expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, for example from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. The protein is then purified from culture media or cell extracts. Any of the polypeptide chains that comprise the dimeric antigen receptors (DAR) or antigen-binding portions thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5): 446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are at least 65% pure, at least 75% pure, at least 85% pure, at least 95% pure, or at least 98% pure. Regardless of the exact numerical value of the purity, the polypeptide is sufficiently pure for use as a pharmaceutical product. Any of the dimeric antigen receptors (DAR) or antigen-binding portions thereof that are described herein can be expressed by transgenic host cells and then purified to about 65-98% purity or high level of purity using any art-known method.

In certain embodiments, the antibodies and antigen binding proteins described herein (e.g., DAR) can further comprise post-translational modifications. Exemplary post-translational protein modifications include phosphorylation, acetylation, methylation, ADP-ribosylation, ubiquitination, glycosylation, afucosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, glycosylation can be sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

In one embodiment, the dimeric antigen receptors (DAR) described herein can be modified to become soluble polypeptides which comprises linking the antibodies and antigen binding proteins to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

The present disclosure provides therapeutic compositions comprising any of the DAR-expressing cells that are described herein in an admixture with a pharmaceutically-acceptable excipient. An excipient encompasses carriers, stabilizers and excipients. Excipients of pharmaceutically acceptable excipients includes for example inert diluents or fillers (e.g., sucrose and sorbitol), salts, buffering agents, stabilizing agents, preservatives, cryoprotectants, non-ionic detergents, anti-oxidants and isotonifiers.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the GD2 DAR-expressing cells described herein may be administered in a solution of a pharmaceutically acceptable salt, such as non-toxic acid addition salts. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. The DAR-expressing cells may be provided in a composition that includes, for example, a cell culture medium, PBS, HBSS, Ringer's, or Tyrode's solution and may include growth factors or other proteins that maintain viability or stability of the cell population or allow for propagation of the DAR-expressing cells.

The term “subject” as used herein refers to human and non-human animals, including vertebrates, mammals, and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein that include GD2 DAR-expressing cells include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the GD2 DAR-expressing cells described herein can be administered to a subject using art-known methods and delivery routes.

The terms “effective amount”, “therapeutically effective amount” or “effective dose” or related terms may be used interchangeably and refer to an amount of any of the DAR-expressing cells described herein that when administered to a subject, is sufficient to effect a measurable improvement or prevention of a disease or disorder associated with tumor or cancer antigen expression. Therapeutically effective amounts of DAR-T or DAR-NK cells provided herein, when used alone or in combination, will vary depending upon the relative activity of the antibodies and combinations (e.g. , in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, cells expressing a DAR (e.g., GD2 DAR-T cells or GD2 DAR-NK cells) are administered to a human subject at about 1×10³-1×10¹² cells/kg with or without prior lymphodepletion. DAR-T cells may be administered daily (e.g., once, twice, three times, or four times daily) or less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

In one embodiment, a therapeutically effective amount comprises a dose of about 103 - 1012 DAR-T cells per kg weight administered to the subject. In one embodiment, the transgenic host cells harbor one or more expression vectors that express the polypeptide chains that comprise any of the DARs described herein. The therapeutically effective amount can be determined by considering the subject to receive the therapeutically effective amount and the disease/disorder to be treated which may be ascertained by one skilled in the art using known techniques. The therapeutically effective amount may consider factors pertaining to the subject such as age, body weight, general health, sex, diet, time of administration, drug interaction, and the severity of the disease/disorder. The therapeutically effective amount may consider the purity of the transgenic host cells, which can be about 65%-98% or higher levels of purity. The therapeutically effective amount of the transgenic host cells can be administered to the subject at least once, or twice, three times, 4 times, 5 times, or more over a period of time. The period of time can be per day, per week, per month, or per year. The therapeutically effective amount of the transgenic cells administered to the subject can be same each time or can be increased or decreased at each administration event. The therapeutically effective amount of the transgenic cells can be administered to the subject until the tumor size or number of cancer cells is reduced by 5%-90% or more, compared to the tumor size or number of cancer cells prior to administration of the transgenic host cells.

The present disclosure provides methods for treating a subject having a

disease/disorder associated with expression or over-expression of one or more tumor-associated antigens. The disease comprises cancer or tumor cells expressing the tumor-associated antigens, such as for example GD2 antigen. In one embodiment, the cancer or tumor includes neuroblastomas, melanomas, small-cell lung cancer, medulloblastomas, astrocytomas, osteosarcomas and other soft tissue sarcomas.

### GD2 Dimeric Antigen Receptors

The present disclosure provides dimeric antigen receptors (DARs) comprising two polypeptides that together comprise a Fab fragment that binds GD2, where the Fab fragment is joined to a single transmembrane region and intracellular regions that are components of the second polypeptide. DARs as provided herein comprise an antibody heavy chain variable region and an antibody light chain variable region on separate polypeptide chains, where the heavy chain variable region and the light chain variable region form an antigen binding domain. The present disclosure provides dimeric antigen receptors (DARs) having first and second polypeptide chains that associate with each other to form an antigen binding domain that binds a GD2 molecule (e.g., target antigen). In one embodiment, GD2 is overexpressed on tumors, for example tumors of neuroectodermal origin including human neuroblastoma and melanoma.

In some embodiments, a GD2 DAR includes an optional hinge region between the Fab fragment and the transmembrane region, where the hinge region is N-terminal to the transmembrane region of the first polypeptide. In some embodiments, host cells expressing a GD2 DAR as provided herein can demonstrate greater specificity in response to GD2-positive target cells, e.g., in clonal expansion, cytokine release, and/or cytotoxicity, as compared to host cells expressing a CAR having an scFv format of the same anti-GD2 antibody.

The present disclosure provides GD2 DARs comprising an antibody heavy chain variable region on one polypeptide chain that includes a transmembrane domain and an antibody light chain variable region on a separate polypeptide chain that does not include a transmembrane domain or a light chain variable region on one polypeptide chain that includes a transmembrane domain and a heavy chain variable region on a separate polypeptide chain that does not include a transmembrane domain. The two polypeptide chains that make up the DARs can dimerize, for example, through their antibody constant regions at the exterior of the cell, to form a protein complex. The DARs have antibody-like properties as they bind specifically to a target antigen. The dimeric antigen receptors can be expressed by cells used for directed cell therapy.

The present disclosure provides transgenic T cells engineered to express GD2 DAR constructs having an antigen-binding extracellular portion, optional hinge portion, transmembrane portion, and an intracellular portion having co-stimulatory and/or intracellular signaling regions. The extracellular portion exhibits high affinity and avidity to bind GD2-expressing diseased cells leading to T cell activation and diseased-cell killing, while sparing normal cells. The intracellular portion of the GD2 DARs comprises co-stimulatory and/or signaling regions that mediate T cell activation upon antigen binding which can lead to enhanced T cell expansion, formation of memory T cells and/or reduced T cells exhaustion. Described herein are multiple configurations of GD2 DARs that differ in the type and number of intracellular co-stimulatory and signaling regions, providing flexibility in designing GD2 DARs for producing a strong and rapid effector response and/or generating a longer-lasting memory T cell population (e.g., GD2 DARs comprising an intracellular 4-1BB co-stimulatory region).

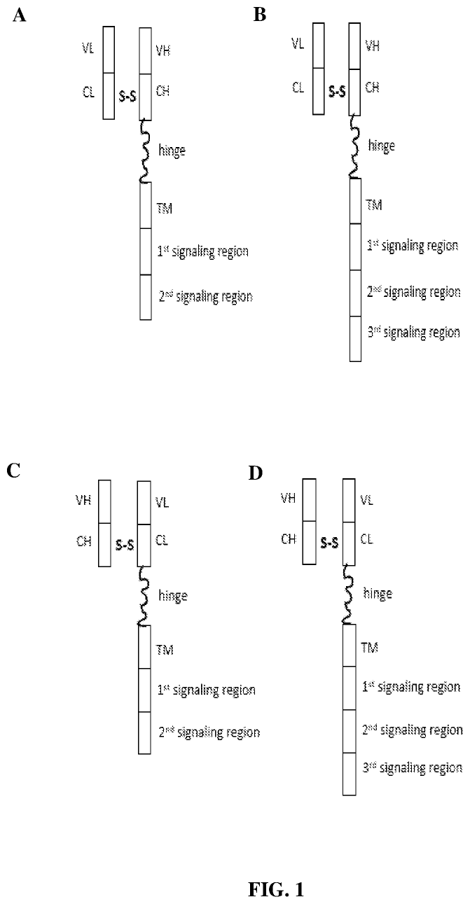

The present disclosure provides a GD2 DAR having a first polypeptide chain and a second polypeptide chain, wherein the first polypeptide chain comprises a heavy chain variable region of an antibody and the second polypeptide chain comprises a light chain variable region of an antibody, wherein the first polypeptide chain is linked to the second polypeptide chain by one or a plurality of disulfide bonds at regions outside of a genetically engineered cell when both the first polypeptide chain and the second polypeptide chain are expressed by the cell. In some embodiments, a GD2 DAR comprises a first polypeptide chain comprising, in sequence, an antibody heavy chain variable domain region and a heavy chain CHI region, an optional hinge region, a transmembrane region, and an intracellular region having 2-5 signaling domains, and a second polypeptide chain comprising an antibody light chain variable domain region with a corresponding light chain constant region (CL), which may be a kappa or lambda light chain constant region, where the CHI and CL regions in each first and second polypeptide chains can be linked with one or two disulfide bonds (e.g., see FIGS. 1A and 1B).

The present disclosure also provides a GD2 DAR having a first polypeptide chain and a second polypeptide chain, wherein the first polypeptide chain comprises a light chain variable region of an antibody and the second polypeptide chain comprises a heavy chain variable region of an antibody, wherein the first polypeptide chain is linked to the second polypeptide chain by one or a plurality of disulfide bonds at regions outside of a transduced cell when both the first polypeptide chain and the second polypeptide chain are expressed by a same cell. In some embodiments, a GD2 DAR construct comprises a first polypeptide chain comprising, in sequence, an antibody light chain variable domain region with a corresponding light chain constant (CL) region, which may be a kappa or lambda light chain constant region, followed by a hinge region, a transmembrane region, and an intracellular region having 2-5 signaling domains, and a second polypeptide chain comprising an antibody heavy chain variable domain region and a CHI region, wherein the CL and CHI regions in the first and second polypeptide chains can be linked with one or two disulfide bonds (e.g., see FIGS. 1C and 1D). In some embodiments, a GD2 DAR construct consists essentially of a first polypeptide chain comprising, in sequence, an antibody light chain variable domain region with a corresponding light chain constant (CL) region, which may be a kappa or lambda light chain constant region, followed by a hinge region, a transmembrane region, and an intracellular region having 2-5 signaling domains, and a second polypeptide chain comprising an antibody heavy chain variable domain region and a CHI region, wherein the CL and CHI regions in the first and second polypeptide chains can be linked with one or two disulfide bonds.

The first polypeptide chain of the GD2 DAR can comprise, for example, an antibody heavy chain variable region comprising an amino acid sequence having at least 95% sequence identity to the amino acid sequence of: mouse 14.18 (14.18) heavy chain variable region according to SEQ ID NO:2; humanized 14.18 (hu14.18) heavy chain variable region according to SEQ ID NO:3; chimeric 3F8 (ch3F8) heavy chain variable region; or humanized 3F8 (hu3F8) heavy chain variable region. The antibody heavy chain constant region comprises sequences derived from a human antibody heavy chain constant region, e.g., a human CHI domain (e.g., SEQ ID NO:4). In various embodiments, the antibody heavy chain constant region can be derived from an IgM, IgA, IgG, IgE or IgD antibody. The second polypeptide chain of the GD2 DAR can comprise, for example, an antibody light chain variable region comprising an amino acid sequence having at least 95% sequence identity to the amino acid sequence of: the mouse 14.18 (14.18) light chain variable region according to SEQ ID NO:5; the humanized 14.18 (hu14.18) light chain variable region according to SEQ ID NO:6; chimeric 3F8 (ch3F8) light chain variable region; or humanized 3F8 (hu3F8) light chain variable region. The antibody light chain constant region can comprise sequences derived from a human antibody light chain constant region, e.g., a human CL domain, that can be a kappa or lambda CL domain (e.g., SEQ ID NO:7 or SEQ ID NO:8).

Alternatively to the above, the first polypeptide chain of the GD2 DAR can comprise, for example, an antibody light chain variable region comprising an amino acid sequence having at least 95% sequence identity to the amino acid sequence of: the mouse 14.18 (14.18) light chain variable region according to SEQ ID NO:5; the humanized 14.18 (hu14.18) light chain variable region according to SEQ ID NO:6; chimeric 3F8 (ch3F8) light chain variable region; or humanized 3F8 (hu3F8) light chain variable region. The antibody light chain constant region can comprise sequences derived from a human antibody light chain constant region, e.g., a human CL domain, that can be a kappa or lambda CL domain (e.g., SEQ ID NO:7 or SEQ ID NO:8). The second polypeptide chain of the GD2 DAR can comprise, for example, an antibody heavy chain variable region comprising an amino acid sequence having at least 95% sequence identity to the amino acid sequence of: mouse 14.18 (14.18) heavy chain variable region according to SEQ ID NO:2; humanized 14.18 (hu14.18) heavy chain variable region according to SEQ ID NO:3; chimeric 3F8 (ch3F8) heavy chain variable region; or humanized 3F8 (hu3F8) heavy chain variable region. The antibody heavy chain constant region comprises sequences derived from a human antibody heavy chain constant region, e.g., a human CHI domain (e.g., SEQ ID NO:4). In one embodiment, the antibody heavy chain constant region can be derived from an IgM, IgA, IgG, IgE or IgD antibody.

A GD2 DAR as provided herein may or may not have a hinge region. In some embodiments, a GD2 DAR comprises a hinge region where the hinge region is about 10 to about 120 amino acids in length. In some nonlimiting examples, the hinge region can be a CD28 hinge region or a fragment thereof (e.g., SEQ ID NO:9), a CD8a hinge region or a fragment thereof (e.g., SEQ ID NO: 10), a hinge region that combines the hinge regions of CD28 and CD8 (e.g., SEQ ID NO:11), or a hinge region of an antibody (IgG, IgA, IgM, IgE, or IgD) that joins the constant domains CHI and CH2 of the antibody, or hinge regions derived from any thereof having at least 95%, 96%, 97%, 98%, or 99% to any of these. A hinge region derived from an antibody may or may not comprise one or more constant regions of the antibody or an amino acid sequence thereof.

In various embodiments, the transmembrane domain can be derived from a membrane protein sequence region selected from the group consisting of CD8α (e.g., SEQ ID NO:13), CD8β, 4-1BB/CD137, CD28 (e.g., SEQ ID NO:12), CD34, CD4, FcϵRIγ, CD16, OX40/CD134, CD3ζ, CD3ϵ, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, and FGFR2B.

In various embodiments, the signaling region can be selected from the group consisting of signaling regions from CD3-zeta chain, 4-1BB (e.g., SEQ ID NO: 16), CD28 (e.g., SEQ ID NO:17), CD27, OX40 (e.g., SEQ ID NO: 18), CD30, CD40, PD-1, ICOS, lymph oocyte function-associated antigen-1 (LFA-1), CD2, CD7, LIGHT, NKG2C, B7-H3, GITR (TNFRSF18), DR3 (TNFRSF25), TNFR2, CD226, and combinations thereof. For example, a signaling region of a DAR first polypeptide can have two or three signaling regions derived from any of these signaling regions. In some embodiments, a GD2 DAR as provided herein has a signaling region that includes a signaling region derived from CD3zeta having ITAM1, ITAM, 2, and/or ITAM3 of the CD3zeta signaling region (SEQ ID NO:19), and has at least one additional signaling region (or co-stimulatory signaling domain) that can be, as nonlimiting examples, a 4-1BB (SEQ ID NO:16), CD28 (SEQ ID NO:17), or OX40 (SEQ ID NO:18) co-stimulatory domain. In some embodiments, the intracellular region comprises a CD28 co-stimulatory region (e.g., SEQ ID NO:17) and CD3-zeta intracellular signaling sequences (e.g., SEQ ID NO:19, SEQ ID NO:20, SEQ ID NO:21, or SEQ ID NO:22), or 4-1BB co-stimulatory and CD3-zeta intracellular signaling sequences. In some embodiments, the CD3-zeta portion of the intracellular signaling region comprises ITAM (immunoreceptor tyrosine-based activation motif) motifs 1, 2 and 3 (e.g., long CD3-zeta). In some embodiments, the CD3-zeta portion of the intracellular signaling region comprises only one of the ITAM motifs such as only ITAM 1, 2 or 3 (e.g., short CD3-zeta).

In one embodiment, the hinge region comprises a CD28 hinge comprising the amino acid sequence of SEQ ID NO:9, or a CD8 hinge comprising the amino acid sequence of SEQ ID NO:10, or a hinge region comprising a CD28 and CD8 hinge sequences of SEQ ID NO:11 (e.g., long hinge). In one embodiment, the first polypeptide lacks a hinge region. In one embodiment, the transmembrane region comprises the amino acid sequence of: SEQ ID NO: 12 (from CD28); SEQ ID NO:13 (from CD8); SEQ ID NO:14 (from 4-1BB); or SEQ ID NO:15 (from CD3zeta). In one embodiment, the intracellular region comprises the amino acid sequence from any one or any combination of two or more intracellular sequences selected from a group consisting of: SEQ ID NO:16 (from 4-1BB); SEQ ID NO:17 (from CD28); SEQ ID NO:18 (from OX40); SEQ ID NO:19 (CD3zeta ITAM 1, 2 and 3); SEQ ID NO:20 (CD3zeta ITAM 1); SEQ ID NO:21 (CD3zeta ITAM 2); and/or SEQ ID NO:22 (CD3zeta ITAM 3). In one embodiment, the first polypeptide chain comprises leader sequence comprising the amino acid sequence of SEQ ID NO:23, 24 or 25, or the first polypeptide lacks a leader sequence.

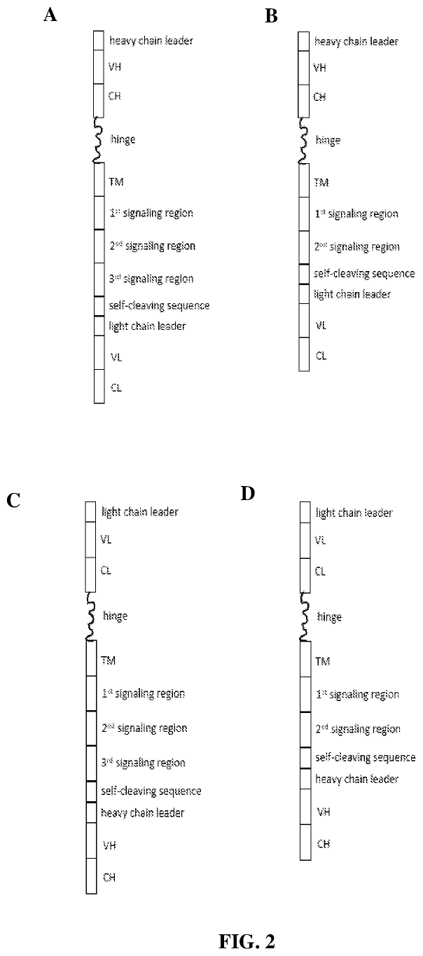

Nucleic acid molecules that encode the GD2 DARs as described herein, including precursor polypeptides of one or both of a first and second DAR polypeptide (e.g., FIG. 2A-D) are also provided. In various embodiments, the GD2 DAR-encoding sequences may be operably linked to a promoter, such as a promoter functional in a eukaryotic cell, in an expression cassette.

Further included are host cells genetically engineered to include a nucleic acid sequence encoding a GD2 DAR as provided herein. The cells in various embodiments can be knocked out for T cell receptor expression, and can be, as nonlimiting examples, T cells or NK cells, such as human T cell or NK cells. The genetically engineered cells may be primary cells. A population of cells, such as T cells or NK cells, that has been genetically engineered to express a GD2 DAR as described herein, can be a population in which at least 10%, at least 20%, at least 30%, at least 40% or at least 50% of the cells of the population express the GD2 DAR. A population of cells, such as T cells or NK cells, that has been genetically engineered to express a GD2 DAR as described herein, can be a population in which at least 10%, at least 20%, at least 30%, at least 40% or at least 50% of the cells of the population express the GD2 DAR and do not express a T cell receptor, e.g., do not express an endogenous T cell receptor.

The host cells provided herein that express a GD2 DAR can be provided as a pharmaceutical composition in a buffer, salt solution, or cell media formulation that comprises, for example, PBS, HBSS, Ringer's, or Tyrode's solution. A composition that includes host cells that express a GD2 DAR as provided herein can further include a cryoprotectant, such as, for example, DMSO, glycerol, or a sugar alcohol and the composition may be provided as a frozen composition. A composition that includes host cells that express a GD2 DAR as provided herein can optionally include proteins, peptides, sugars, lipids, polymers, antioxidants, enzymes, small molecules or other compounds that may contribute to the stability, viability, or functionality of the cells and/or may include one or more compounds that may provide therapuetic benefit, including but not limited to antibodies (including engineered polypeptides having antibody domains), cytokines, growth factors, or small molecules.

Host cells expressing a GD2 DAR as provided herein may be used in methods of treating a subject with cancer, such as, for example, a neuroblastoma, melanoma, small-cell lung cancer, medulloblastoma, astrocytoma, or osteosarcoma. The T cells, which may lack endogenous T cell receptor expression, in some embodiments may be allogeneic with respect to the subject being treated. The methods can comprise administering to the subject an effective amount of a population of host cells that include at least one nucleic acid sequence encoding a GD2 DAR as provided herein. Administration can be via any practical route. In some examples, the administration is an intravenous administration. In some examples, administration can include injection, such as intratumoral or peritumoral injection. In some embodiments, one or multiple dosages of the GD2 DAR T-cell population can be administered after the onset or detection of a cancer and optionally for a length of time necessary for the treatment of the disease.

### EXAMPLES

The following examples are meant to be illustrative and can be used to further

understand embodiments of the present disclosure. They should not be construed as limiting the scope of the present teachings in any way.

### Example 1

### Expression of GD2 on Tumor Cell Lines

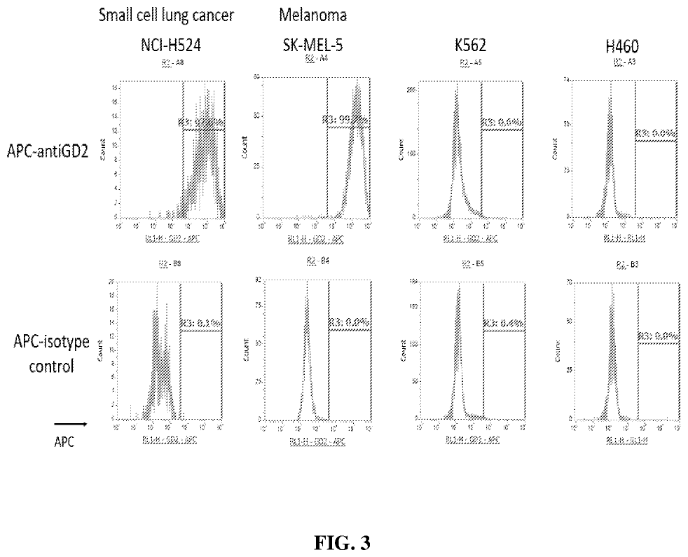

Flow cytometry was used to detect GD2 on cell lines NCI-H524 (GD2+) (small cell lung cancer); SK-MEL-5 (GD2+) (melanoma); K562 (GD2−) (erythroleukemia (CML)); and H460 (GD2+) (non-small cell lung cancer) using an anti-GD2 antibody conjugated to allophycocyanin (APC). In control experiments cells were incubated with an IgG conjugated to APC. FIG. 3 demonstrates that H524 and SK-MEL-5 have detectable GD2 expression, while K562 and H460 have low or no detectable GD2 on the cell surface.

### Example 2

### Isolation of Human PBMC Cells and Primary T Cells

Primary human T cells were isolated from healthy human donors either from buffy coats (San Diego blood bank), fresh blood or leukapheresis products (StemCell Technologies, Vancouver, CA). Peripheral blood mononuclear cells (PBMCs) were isolated by density gradient centrifugation. In some experiments, PBMCs rather than isolated T cells were transfected with GD2 DAR and CAR constructs.

T cells were isolated from PBMCs by magnetic negative selection using EASYSEP Human T Cell Isolation Kit (StemCell Technologies) or by positive selection and activation by DYNABEADS Human T-Expander CD3/CD28 (Thermo Fisher Scientific, Waltham, MA, USA) according to manufacturer's instructions.

In some transfections T cells were expanded from PBMCs after monocytes were removed by plating PBMCs in a coated cell culture flask for one to two hours, after which the nonadherent lymphocytes were washed from the flask and then activated with T cell TRANSACT (Miltenyi Biotec, Bergisch Gladbach, Germany) in a new flask according to manufacturer's instructions.

Isolated T cells or T cell populations expanded from PBMCs from which monocytes were removed were either freshly isolated or thawed from frozen storage for transfection with CAR or DAR constructs. The cells were cultured in CTS OPTMIZER T Cell Expansion SFM supplemented with 5% CTS Immune Cell SR (Thermo Fisher Scientific) with 300 U/mL IL-2 (Proleukin) at a density of 10⁶ cells per mL. The cells were activated with 3 uL/10cells per mL T Cell TRANSACT (containing CD3 and CD28 agonists, Miltenyi Biotec) for two to three days prior to transfection with nucleic acid molecules encoding either a precursor anti-GD2 CAR or a precursor anti-GD2 DAR.

### Example 3

### Preparation of T Cell Receptor Knockout/DAR-T Cells

Activated T cells (approximately 9×10⁶ (9e6) cells) or in some cases PBMCs were transfected with nucleic acids encoding either a GD2 DAR or a GD2 CAR. CRISPR technology was used to direct insertion of the CAR or DAR construct into the TRAC (T cell receptor alpha constant) gene (also known as the TRA gene, NCBI Gene ID:6955) such that the T cell receptor was knocked out in cells in which the engineered GD2 receptor was stably integrated into the genome and expressed on the cell surface.

The two GD2 DAR constructs that were introduced into the primary T cells or PBMCs differed only in the sequences of the antibody domains of the first and second polypeptide chains of the DARs. The GD2-14.18 DAR included heavy and light chain variable regions of anti-GD2 antibody 14.18 (Mujoo et al. (1989) Cancer Research 49:2857-2861). The GD2-14.18 DAR first polypeptide included the anti-GD2 monoclonal antibody 14.18 heavy chain variable region (SEQ ID NO:2) linked to a human heavy chain constant region (SEQ ID NO:4) and the GD2-14.18 DAR second polypeptide included the anti-GD2 monoclonal antibody 14.18 light chain variable region (SEQ ID NO:5) linked to a human kappa light chain constant region (SEQ ID NO:7). The GD2-hu14.18 DAR included heavy and light chain variable regions of the humanized anti-GD2 antibody hu14.18 (U.S. Pat. No. 7,169,904). The GD2-hu14.18 DAR first polypeptide included the anti-GD2 humanized antibody hu14.18 heavy chain variable region (SEQ ID NO:3) linked to a human heavy chain constant region (SEQ ID NO:4) and the GD2-hu14.18 DAR second polypeptide included the anti-GD2 humanized antibody hu14.18 light chain variable region (SEQ ID NO:6) linked to a human kappa light chain constant region (SEQ ID NO:7).

Both of these GD2 DARs had identical configurations (FIG. 1A) and identical hinge, transmembrane, and intracellular regions.

The GD2-14.18 DAR first polypeptide included, from the N-terminus to the C-terminus, the 14.18 antibody heavy chain variable region of SEQ ID NO:2 followed by the heavy chain CHI region (SEQ ID NO:4), the hinge region of CD28 (SEQ ID NO:9), the transmembrane region of CD28 (SEQ ID NO: 12), the co-stimulatory intracellular domain of 4-1BB (SEQ ID NO:16), and the internal signaling domain of CD35 that included ITAMs 1, 2, and 3 (SEQ ID NO: 19). The second polypeptide of the GD2-14.18 DAR included, from the N-terminus to the C-terminus, the 14.18 antibody light chain variable region of SEQ ID NO:5 followed by the light chain CL kappa region (SEQ ID NO:7). The nucleic acid construct encoding the first and second polypeptides included sequences encoding signal peptides at the N-termini of the first and second polypeptides for synthesis of precursor polypeptides for integration into the membrane of host cells and secretion from host cells respectively: a sequence encoding SEQ ID NO:23 preceded the sequence encoding the VH domain of the first polypeptide and a sequence encoding SEQ ID NO:24 preceded the sequence encoding the VL domain of the second polypeptide. The two precursor polypeptides including the signal peptides were encoded by a single transcriptional unit in which the first polypeptide-encoding sequence and second polypeptide-encoding sequence were linked by a sequence encoding the T2A amino acid sequence (SEQ ID NO:26) such that the two polypeptides encoded by a single transcript would be translated and processed into two mature polypeptides (SEQ ID NO:32 and SEQ ID NO:33) that would assemble into a DAR via disulfide bonds through their antibody constant regions at the outside of the cell. The nucleic acid sequence encoding the two precursor polypeptides linked by the T2A sequence (SEQ ID NO:30) is provided as SEQ ID NO:31. The nucleic acid sequence of SEQ ID NO:31 was operably linked to a JeT promoter (SEQ ID NO:39) to provide an expression cassette that was transfected into T cells.

The second GD2 DAR construct, encoding GD2-hu14.18 DAR, included heavy and light chain variable regions of anti-GD2 antibody hu14.18. The GD2-hu14.18 DAR first polypeptide included, from the N-terminus to the C-terminus, the hu14.18 heavy chain variable region of SEQ ID NO:3 followed by the heavy chain CH1 region (SEQ ID NO:4), the hinge region of CD28 (SEQ ID NO:9), the transmembrane region of CD28 (SEQ ID NO:12), the co-stimulatory intracellular domain of 4-1BB (SEQ ID NO:16), and the internal signaling domain of CD35 that included ITAMs 1, 2, and 3 (SEQ ID NO: 19). The second polypeptide of the GD2-hu14.18 DAR included, from the N-terminus to the C-terminus, the hu14.18 light chain variable region of SEQ ID NO:6 followed by the light chain CL kappa region (SEQ ID NO:7). The nucleic acid construct encoding the first and second polypeptides included sequences encoding signal peptides at the N-terminus of the first and second polypeptides for synthesis of precursor polypeptides for integration into the membrane of host cells and secretion from host cells respectively: a sequence encoding SEQ ID NO:23 preceded the sequence encoding the VH domain of the first polypeptide and SEQ ID NO:24 preceded the sequence encoding the VH domain of the second polypeptide. The two precursor polypeptides including the signal peptides were encoded by a single transcriptional unit in which the first polypeptide-encoding sequence and second polypeptide-encoding sequence were linked by a sequence encoding T2A amino acids (SEQ ID NO:26) such that the two polypeptides encoded by a single transcript would be translated and processed into two mature polypeptides (SEQ ID NO:36 and SEQ ID NO:37) that would assemble into a DAR via disulfide bonds through their antibody constant regions at the outside of the cell. The nucleic acid sequence encoding the two polypeptides of the GD2 hu14.18 DAR linked by the T2A sequence is provided as SEQ ID NO:35. The nucleic acid sequence of SEQ ID NO:35 was operably linked to a JeT promoter (SEQ ID NO:39) to provide an expression cassette that was transfected into T cells.

GD2 CAR constructs were also generated in which an scFv based on the anti-GD2 14.18 antibody heavy and light chain sequences of SEQ ID NO:2 and SEQ ID NO:5, joined by a GS linker (SEQ ID NO:38) were linked to a CD28 hinge region (SEQ ID NO:9), CD28 transmembrane region (SEQ ID NO:12), and CD28 co-stimulatory domain (SEQ ID NO:17), and a CD35 intracellular signaling domain (SEQ ID NO:19). Another construct included the hu14.18 antibody heavy and light chain sequences of SEQ ID NO:3 and SEQ ID NO:6 , joined by the same GS linker (SEQ ID NO:38), and also linked to the CD28 hinge region (SEQ ID NO:9), CD28 transmembrane region (SEQ ID NO:12), CD28 co-stimulatory domain (SEQ ID NO:17), and CD3 intracellular signaling domain (SEQ ID NO:19). The constructs encoding these CARs were also linked to the JeT promoter (SEQ ID NO:39) in expression cassettes for transfection into primary T cells.

The Cas9 RNA-guided endonuclease was used to generate GD2 DAR-T cells and GD2 CAR-T cells with simultaneous knockout of the TRAC gene. The GD2 DAR and CAR constructs described above cloned downstream of the JeT promoter (SEQ ID NO:39) were cloned into a vector between 5′ and 3′ homology regions of the T cell receptor alpha constant (TRAC) gene (Entrez Gene ID: 28755) (SEQ ID NO:40 and SEQ ID NO:41, respectively) that flanked the target site for Cas9-mediated integration (SEQ ID NO:42) in AAV vector pAAV-MCS. Bacterial clones containing the GD2 DAR or CAR constructs operably linked to the JeT promoter and flanked by the TRAC gene homology regions were confirmed by sequencing.

For CAR or DAR knock-in/TCR knockout using Cas9, the RNP complex was made by first combining an Alt-RR CRISPR-Cas9 crRNA that included the target sequence of SEQ ID NO:42 and an Alt-RR CRISPR-Cas9 tracrRNA (both from IDT, Coralville, IA) and heating the mixture at 95° C. for 5 min. The mixture was then allowed to cool to room temperature (18-25° C.) on the bench top for approximately 20 min to make a crRNA:tracrRNA duplex. For each transfection, 10 μg wild type SpCas9 protein that included nuclear localization sequences (IDT) was mixed with 200 pmol crRNA:tracrRNA duplex and the mixture was incubated at 4° C. for 30 min to form RNPs.

The donor DNA for Cas9-mediated insertion of the GD2 DAR constructs were generated from a pAAV plasmids that included either the GD2-14.18 DAR construct of SEQ ID NO:31 or the GD2-hu14.18 DAR construct of SEQ ID NO:35 flanked by 5′ and 3′ homology sequences (SEQ ID NO:40 and SEQ ID NO:41, respectively) from the TRAC gene. The donor fragments having the shorter homology arms of SEQ ID NO:45 (171 bp) and SEQ ID NO:46 (161 bp) was produced using a forward primer having the sequence: A*TmC*mA*mCGAGCAGCTGGTTTCT (SEQ ID NO: 43), and a reverse primer having the sequence: GACCTCATGTCTAGCACAGTTTTG (SEQ ID NO:44). The forward primer (SEQ ID NO:43) included phosphorothioate bonds between the first and second, third and fourth, and fourth and fifth nucleotides from the 5′ terminus (designated with an asterisk (*)). The nucleotides at the third, fourth, and fifth positions from the 5′-end of the forward oligonucleotide primer were 2′-O-methyl modified (designated as mC, mA, and mC). The reverse primer (SEQ ID NO:44) did not have any chemical modifications but included a 5′-terminal phosphate. PCR was performed essentially as provided above to produce double-stranded donor DNA molecules having a GD2 DAR expression cassette flanked by TRAC locus homology arms of 171 and 161 bps (SEQ ID NO:45 and SEQ ID NO:46). The resulting double stranded GD2 DAR donor DNA fragments were used in independent transfections of activated T cells as double-stranded molecules together with the Cas9 RNP.

The donor DNAs that included the GD2 CAR constructs were designed and synthesized in the same way as the donors that included the GD2 DAR constructs. The same primers (SEQ ID NO:43 and SEQ ID NO:44) were used to generate the double-stranded donor fragment with 171 and 161 bp homology arms.

Transfection of cells with GD2 CAR and GD2 DAR donor DNAs plus a Cas9 RNP targeting exon 1 of the TRAC locus, was performed essentially as described in US 2020/0224160; WO 2020/176740, and WO 2020/185867, all of which are incorporated by reference herein in their entireties. Following electroporation with Cas9 RNP and donor fragment, cells were diluted into culture medium and incubated at 37° C., 5% CO₂ in OpTmizer™ T Cell Expansion SFM supplemented with 5% serum replacement and 300U/ml IL2 in 37° C. Once cells were in expansion cultures, cell counts were obtained every 2 or 3 days and the cell concentration was maintained at 5×10⁵ (5e5) to 1×10⁶ (1e6) per mL.

For use as control cells, isolated T cells were transfected with the TRAC-targeting RNP in the absence of a donor fragment. Such cells had a disrupted TRAC gene but no insertion of a GD2 DAR or CAR construct and are thus TCR knockout (TRAC KO) cells.

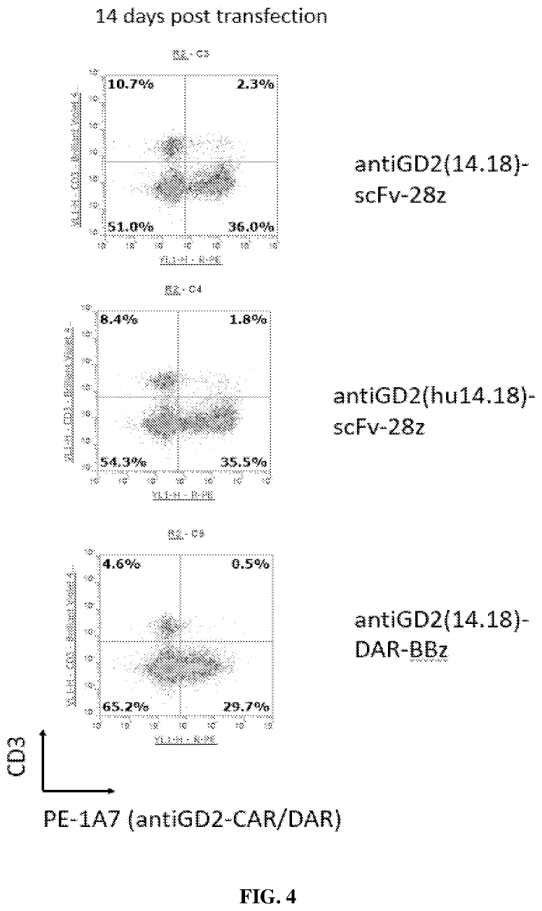

FIG. 4 provides the results of flow cytometry using a labeled antibody to CD3 for detection of the T cell receptor and anti-idiotypic antibody 1A7 that mimics GD2 for detection of a GD2 DAR or CAR after fourteen days in culture. Cells transfected with the two versions of GD2 CAR had approximately 36% of the population expressing the GD2 CAR (x axis) in the absence of T cell receptor expression (y axis). The GD2(14.18) DAR-transfected cell population had a somewhat lower percentage of cells expressing GD2 DAR in the absence of T cell receptor expression (approximately 30%). Nearly all of the cells expressing the GD2 DAR did not express the T cell receptor (i.e., were CD3 negative) (lower panel).

### Example 3

### In Vitro Expansion of CAR-T and DAR-T Cells

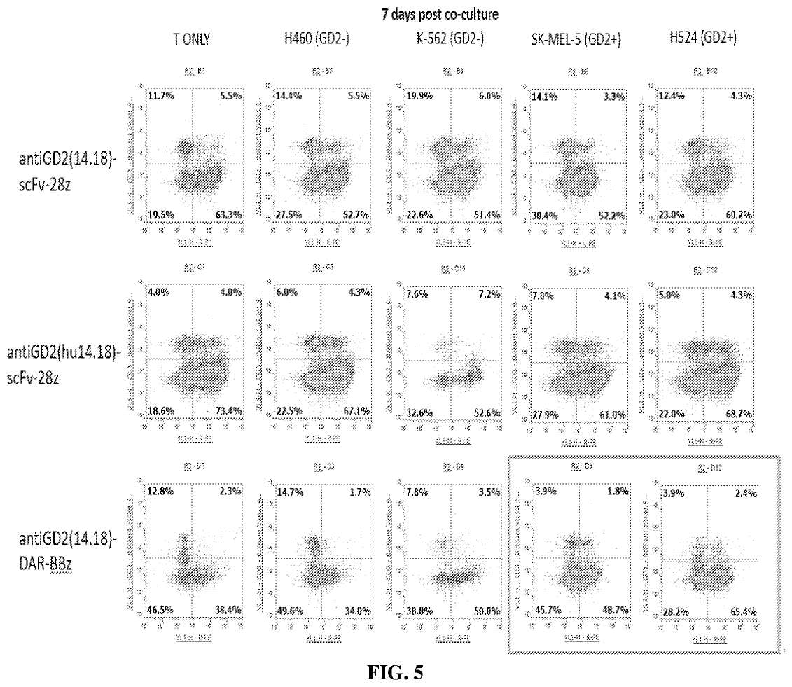

Clonal expansion of GD2 CAR-T and DAR-T cells was tested on cells that expressed GD2 (GD2+) and as controls, cells that had no detectable expression of GD2 (GD2−) (FIG. 3). The GD2 CAR and DAR transfected cell populations were co-cultured separately with NCI-H542 GD2+ cells, and SK-MEL-5 GD2+ cells, K562 GD2− cells, and H460 GD2- cells. The level of cell expansion was measured using flow cytometry after 7 days of co-culture (FIG. 5), when the percentage of the T cell population expressing the GD2 DAR or CAR in the absence of T cell receptor expression was assessed essentially as in Example 2. FIG. 5 shows that when compared with the percentage of the population expressing the GD2 DAR or CAR cultured for the same amount of time in the absence of co-culture cells (leftmost panels), the percentage of DAR-T cells increased with co-culture with GD2+ cells, while the same was not true for CAR-T cells, which did not increase their share of the T cell population after 7 days of co-culturing with GD+ cells.

### Example 4

### In Vitro Cytotoxicity Assays

For in vitro cytotoxicity assays, cell lines NCI-H524 (small cell lung cancer), SK-MEL-5 (melanoma) and H460 (non-small cell lung cancer) were transduced using a lentivirus carrying luciferase and GFP genes. A single cell clone with luciferase and GFP expression was selected (NCI-H524-Fluc-GFP/puro and SK-MEL-5-Fluc-GFP/puro) and expanded for use in the assays.

The anti-GD2 CAR-T or DAR-T cells were co-cultured with GD2+ NCI-H524-Fluc/GFP/puro or SK-MEL-5-Fluc-GFP/puro cells, or GD2- H460-Fluc-GFP/puro cells. The ratio of effector to target cell ranged from 0.3:1 to 3:1 using H524 cells as targets and from 10:1 to 1.25:1 using SK-MEL-5 cells and H460 cells as targets. After overnight incubation, the cells were subjected to flow cytometry to measure the GFP-expressing cell population to determine the specific target cell killing by anti-GD2 CAR or DAR T cells.

A luciferase-based assay was performed in 96-well plates. 100 μl firefly luciferase labelled NCI-H524 cells were added at 5×10⁵ cells/ml and 100 μl T cells were added to obtain E:T ratios of 3:1, 1:1 and 0.3:1. For tumor only controls 100 μl medium was added and for full lysis control 100 μl medium containing 0.5% Triton X-100 was added. The plate was incubated 24 hours after adding T cells, at which time 75 μl of the assay cultures were transferred into the wells of a black wall 96-well plate (Corning) and mixed with 75 μl ONE-Glo luciferase reagent (Promega). Five minutes after incubation, luminescence was measured using microplate reader (BioTek) and cytotoxicity was calculated after normalizing to tumor only control and full lysis control.

Assays using SK-MEL-5 and H460 cells as targets were performed using the xCelligence Real-time Cell Analyzer Assay. In E-Plate View 96 (Acea Biosciences), SK-MEL-5 and H460 cells were seeded at 1×10⁴ cells per well and the plate was returned to xCELLigence RTCA MP instrument (Acea Biosciences). After overnight incubation, the medium was changed to 100 μl fresh medium and 100 μl T cells were added to get E:T ratios of 10:1, 5:1, 2.5:1 and 1.25:1. For tumor only control 100 μl medium was added and for full lysis control 100 μl medium containing 0.5% Triton X-100 was added. The plate was returned to xCELLigence RTCA MP instrument (Acea Biosciences) for real-time impedance monitoring. Cytotoxicity was calculated using RTCA software pro (Acea Biosciences).

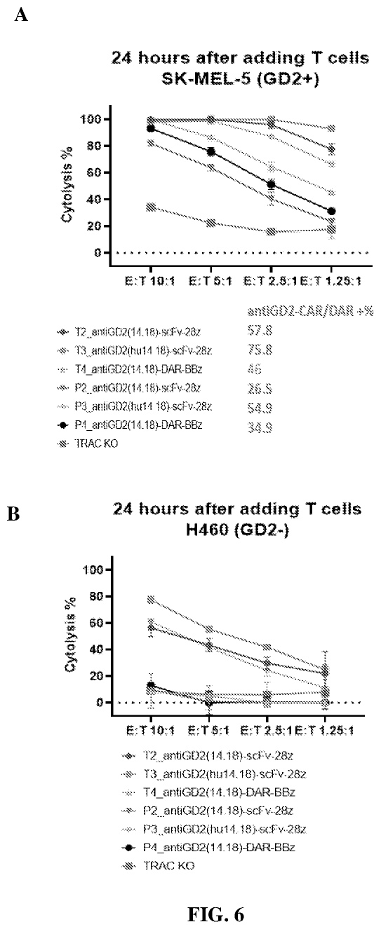

GD2 DAR-T cells prepared by transfecting isolated T cells as well as those prepared by transfecting PBMCs killed H524 (GD2+) target cells and also killed SK-MEL-5 (GD2+) target cells in a dose-dependent manner (FIG. 6A), while exhibiting no cytotoxicity toward GD2− H460 cells (FIG. 6B). Most of the CAR-T cell preparations however exhibited cytotoxicity toward GD2− H460 cells (FIG. 6B).

### Example 5

### In Vitro Cytokine Secretion Assays

GD2 CAR-T cells, GD2 DAR-T cells, and control T cells were subjected to nutrient starvation overnight with IL-2 and then co-cultured with NCI-H524 (GD2+) or K562 (GD2−) cells. After 40 hours incubation, the cells were centrifuged to collect the supernatant for detecting cytokine IFN-gamma (ELISA MAX Delux Set, BioLegend) or GM-CSF (Human GM-CSF Uncoated ELISA kit from Invitrogen/Thermo Fisher) according to the manufacturer's instructions.

In a 96-well plate, 100 μl NCI-H524 or K-562 cells at 5×10⁵ cells/ml were added and for T cell only 100 μl medium were added. 100 μl T cells at 1.5×10⁶ cells/ml were added to get E:T ratio 3:1. The plate was return to incubator for incubation. 48 hours after incubation supernatants were collected and the levels of IFN-gamma and GM-SCF were measured by ELISA.

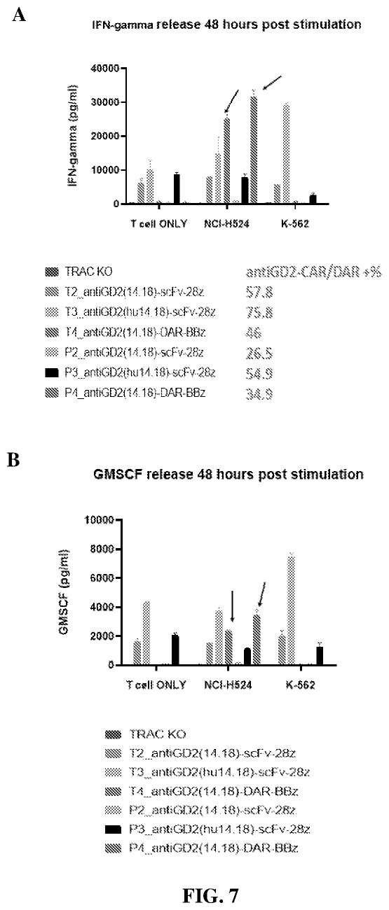

FIG. 7A shows the amount of IFN-gamma secreted by cells transfected with GD2 DAR and GD2 CAR constructs cultured alone or with NCI-H524 cells or K562 cells. Both T cells and PBMCs transfected with the GD2(14.18) DAR construct expressed a high amounts of IFN-gamma when cultured with NCI-H524 (GD2+) cells but had neglible IFN-gamma when cultured with K562 (GD2−) cells. Although one GD2(hu14.18) CAR-T cell population showed a significant amount of IFN-gamma production on NCI-H524 (GD2+) cells, these CAR-T cells produced an even larger amount of IFN-gamma when cultured with K562 (GD2−) cells and also produced a significant amount of IFN-gamma when cultured alone.

FIG. 7B shows the amount of GM-CSF secreted by cells transfected with GD2 DAR and GD2 CAR constructs cultured alone or with NCI-H524 cells or K562 cells. Both T cells and PBMCs transfected with the GD2(14.18) DAR construct expressed IFN-gamma when cultured with NCI-H524 (GD2+) cells but produced undetectable amounts of GM-CSF when cultured with K562 (GD2-) cells. The GD2(hu14.18) CAR-T cell population again showed a significant amount of GM-CSF production on NCI-H524 (GD2+) cells, but as for IFN-gamma production, these CAR-T cells produced an even larger amount of IFN-gamma when cultured with K562 (GD2-) cells and also produced a significant amount of IFN-gamma when cultured alone.

### Example 6

### In Vivo Tumor Killing in a Mouse Model

Tumoricidal activity of the anti-GD2 CAR or DAR T cells was tested in a xenograft mouse model using the SK-MEL-5 (GD2+) tumor cell line that expressed luciferase (tumor-Fluc). A total of approximately 3×10⁶ cells of the tumor-Fluc cells were suspended in 100 μL PBS and then injected subcutanously into the right flank of each mouse.

After 15 days, a single treatment of either 5×10⁷ (5×10⁶ GD2 DAR-positive) anti-GD2−hu14.18 DAR T cells or 5×10⁷ (1.25 x 107 GD2 DAR-positive) anti-GD2-14.18 DAR T cells were administered via the tail vein in 200 μL of PBS. The same amount of TRAC knockout T cells were administered intravenously to a control group and a fourth group received 200 μL of PBS only.

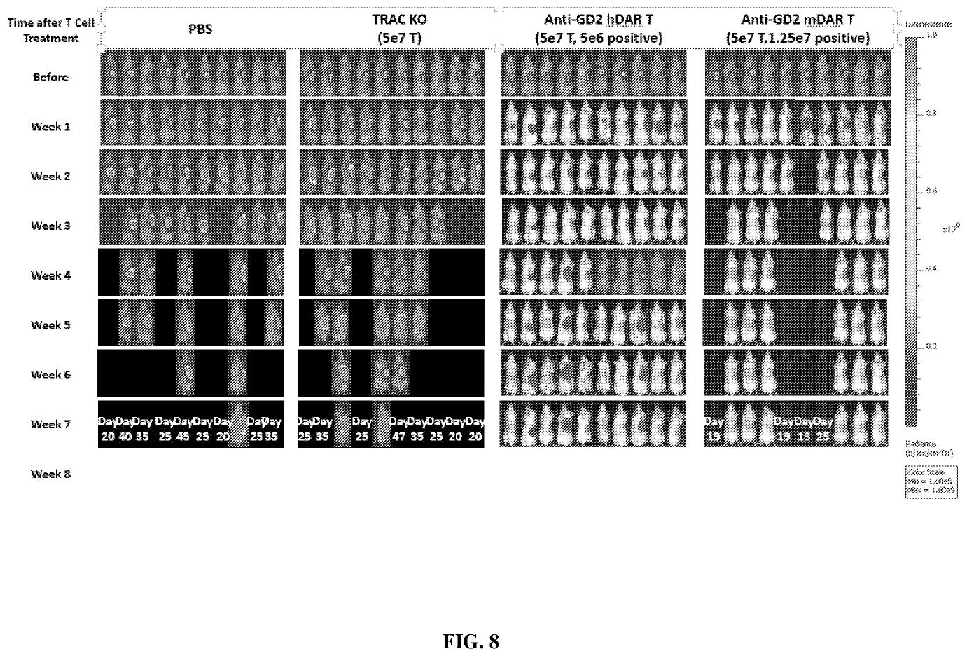

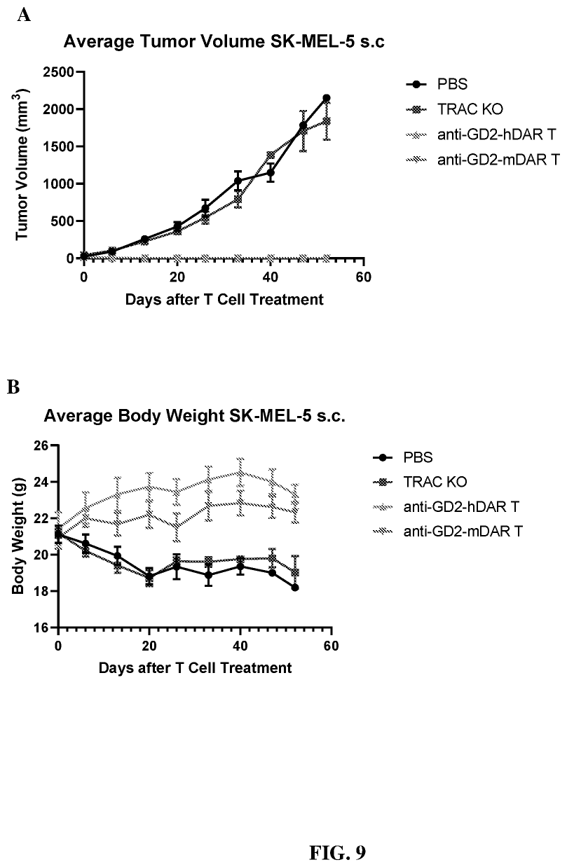

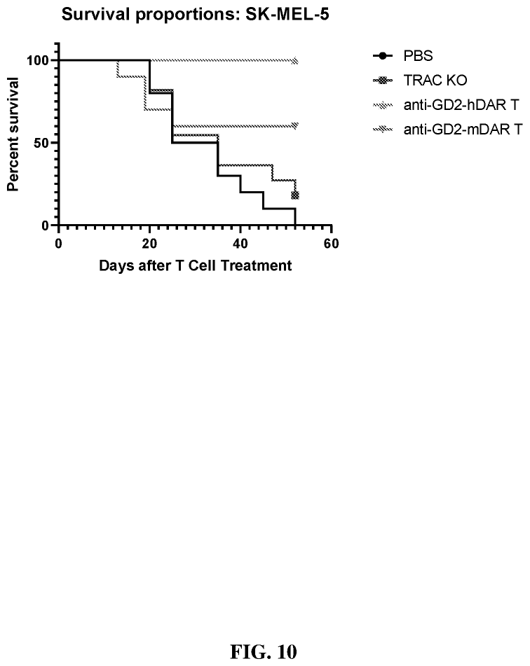

The tumor burden in each animal was monitored using bioluminescence from IVIS imaging as shown in FIG. 8. Tumor volumes and animal bodyweights over time are provided in FIG. 9, showing the GD2 DAR-treated mice had very little establishment of the tumor, in contrast to the control groups, in which the tumor grew steadily over the course of the experiment and did not experience the weight loss exhibited by the control groups. FIG. 10 shows that all of the mice of the GD2-hu14.18 DAR-T cell treatment group survived to the end of the study (approximately 8 weeks), a greater survivorship than any other group. Survivorship of the GD2-14.18 DAR-T cell treatment group was 60% at the end of the study, whereas PBS-treatment and TRAC knockout treatment resulted in only 10% and 20% survivorship, respectively.

SEQUENCES
beta-1,4 N-acetylgalactosaminyltransferase 1 isoform 1 precursor
-UniProtKB Q00973 (B4GN1 HUMAN)
SEQ ID NO: 1
MWLGRRALCALVLLLACASLGLLYASTRDAPGLRLPLAPWAPPQSPRRPELPDLAPEPRYAHIPVRIKEQ
VVGLLAWNNCSCESSGGGLPLPFQKQVRAIDLTKAFDPAELRAASATREQEFQAFLSRSQSPADQLLIAP
ANSPLQYPLQGVEVQPLRSILVPGLSLQAASGQEVYQVNLTASLGTWDVAGEVTGVTLTGEGQADLTLVS
PGLDQLNRQLQLVTYSSRSYQTNTADTVRESTEGHEAAFTIRIRHPPNPRLYPPGSLPQGAQYNISALVT
IATKTFLRYDRLRALITSIRRFYPTVTVVIADDSDKPERVSGPYVEHYLMPEGKGWFAGRNLAVSQVTTK
YVLWVDDDEVFTARTRLERLVDVLERTPLDLVGGAVREISGFATTYRQLLSVEPGAPGLGNCLRQRRGFH
HELVGFPGCVVTDGVVNFFLARTDKVREVGFDPRLSRVAHLEFELDGLGSLRVGSCSDVVVDHASKLKLP
WTSRDAGAETYARYRYPGSLDESQMAKHRLLFFKHRLQCMTSQ
Anti-GD2 14.18 VH
SEQ ID NO: 2
EVQLLQSGPELEKPGASVMISCKASGSSFTGYNMNWVRQNIGKSLEWIGAIDPYYGGTSYNQKFKGRATL
TVDKSSSTAYMHLKSLTSEDSAVYYCVSGMEYWGQGTSVTVSS
Anti-GD2 hu14.18 VH
SEQ ID NO: 3
EVQLVQSGAEVEKPGASVKISCKASGSSFTGYNMNWVRQNIGKSLEWIGAIDPYYGGTSYNQKFKGRATL
TVDKSTSTAYMHLKSLRSEDTAVYYCVSGMEYWGQGTSVTVSS
Anti-GD2 CH1
SEQ ID NO: 4
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHT
Anti-GD2 14.18 VL
SEQ ID NO: 5
EIVMTQSPATLSVSPGERATLSCRSSQSLVHRNGNTYLHWYLQKPGQSPKLLIHKVSNRFSGVPDRESGS
GSGTDFTLKISRVEAEDLGVYFCSQSTHVPPLTFGAGTKLELK
Anti-GD2 hu14.18 VL
SEQ ID NO: 6
DVVMTQTPLSLPVTPGEPASISCRSSQSLVHRNGNTYLHWYLQKPGQSPKLLIHKVSNRFSGVPDRESGS
GSGTDFTLKISRVEAEDLGVYFCSQSTHVPPLTFGAGTKLELK
Anti-GD2 14.18 CL (kappa)
SEQ ID NO: 7
RTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSENRGEC
Anti-GD2 ch14.18 CL (lambda)
SEQ ID NO: 8
GQPKAAPSVTLFPPSSEELQANKATLVCLISDFYPGAVTVAWKADSSPVKAGVETTTPSKQSNNKYAASS
YLSLTPEQWKSHRSYSCQVTHEGSTVEKTVAPTECS
CD28 hinge
SEQ ID NO: 9
PRKIEVMYPPPYLDNEKSNGTIIHVKGKHLCPSPLFPGPSKP
CD8 hinge
SEQ ID NO: 10
AKPTTTPAPRPPTPAPTIASQPLSLRPEACRPAAGGAVHTRGLDFAPR
CD8 hinge + CD28 hinge (long hinge)
SEQ ID NO: 11
AKPTTTPAPRPPTPAPTIASQPLSLRPEACRPAAGGAVHTRGLDFAPRKIEVMYPPPYLDNEKS
NGTIIHVKGKHLCPSPLFPGPSKP
CD28 transmembrane
SEQ ID NO: 12
FWVLVVVGGVLACYSLLVTVAFIIFWV
CD8 transmembrane
SEQ ID NO: 13
IYIWAPLAGTCGVLLLSLVITLY
4-1BB transmembrane
SEQ ID NO: 14
IISFFLALTSTALLFLLFFLTLRESVV
CD3zeta transmembrane
SEQ ID NO: 15
LCYLLDGILFTYGVILTALED
4-1BB co-stimulatory sequence
SEQ ID NO: 16
KRGRKKLLYIFKQPFMRPVQTTQEEDGCSCRFPEEEEGGCEL
CD28 co-stimulatory sequence
SEQ ID NO: 17
RSKRSRLLHSDYMNMTPRRPGPTRKHYQPYAPPRDFAAYRS
OX40 co-stimulatory sequence
SEQ ID NO: 18
ALYLLRRDORLPPDAHKPPGGGSFRTPIQEEQADAHSTLAKI
CD3zeta ITAM 1, 2, 3
SEQ ID NO: 19
RVKFSRSADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
CD3zeta ITAM 1
SEQ ID NO: 20
RVKFSRSADAPAYQQGQNQLYNELNLGRREEYDVLDKR
CD3zeta ITAM 2
SEQ ID NO: 21
RVKFSRSADRGRDPEMGGKPRRKNPQEGLYNELQKDKMAEAYSEIGM
CD3zeta ITAM 3
SEQ ID NO: 22
RVKFSRSADKGERRRGKGHDGLYQGLSTATKDTYDALHMQALPPR
leader sequence
SEQ ID NO: 23
MEWSWVFLFFLSVTTGVHS
Light chain leader sequence
SEQ ID NO: 24
MSVPTQVLGLLLLWLTDARC
Alternative leader sequence
SEQ ID NO: 25
MALPVTALLLPLALLLHAARP
T2A self-cleaving sequence
SEQ ID NO: 26
GSGEGRGSLLTCGDVEENPGP
P2A self-cleaving sequence
SEQ ID NO: 27
GSGATNFSLLKQAGDVEENPGP
E2A self-cleaving sequence
SEQ ID NO: 28
GSGQCTNYALLKLAGDVESNPGP
F2A self-cleaving sequence
SEQ ID NO: 29
GSGVKQTLNFDLLKLAGDVESNPGP
Precursor: GD2-14.18 DAR (version V2a):
SEQ ID NO: 30
MEWSWVFLFFLSVTTGVHSEVOLLQSGPELEKPGASVMISCKASGSSFTGYNMNWVRQNIGKSLEWIGAI
DPYYGGTSYNQKFKGRATLTVDKSSSTAYMHLKSLTSEDSAVYYCVSGMEYWGQGTSVTVSSASTKGPSV
FPLAPSSKSTSGGTAALGCLVKDYFPEPVTVSWNSGALTSGVHTFPAVLQSSGLYSLSSVVTVPSSSLGT
QTYICNVNHKPSNTKVDKRVEPKSCDKTHTPRKIEVMYPPPYLDNEKSNGTIIHVKGKHLCPSPLEPGPS
KPFWVLVVVGGVLACYSLLVTVAFIIFWVKRGRKKLLYIFKQPFMRPVQTTQEEDGCSCRFPEEEEGGCE
LRVKFSRSADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPRGSGEGRGSLLTCGDVEENPGPMSVPTQ
VLGLLLLWLTDARCEIVMTQSPATLSVSPGERATLSCRSSQSLVHRNGNTYLHWYLQKPGQSPKLLIHKV
SNRFSGVPDRESGSGSGTDFTLKISRVEAEDLGVYFCSQSTHVPPLTFGAGTKLELKRTVAAPSVFIFPP
SDEQLKSGTASVVCLLNNFYPREAKVQWKVDNALQSGNSQESVTEQDSKDSTYSLSSTLTLSKADYEKHK
VYACEVTHQGLSSPVTKSENRGEC
Nucleic acid sequence, GD2-14.18 DAR precursor,
SEQ ID NO: 31
ATGGAATGGAGCTGGGTGTTCCTCTTCTTCCTCAGCGTGACCACCGGCGTGCACTCCGAGGTGCAATTAT
TACAGTCCGGACCCGAGCTGGAGAAGCCCGGCGCCTCCGTGATGATCAGCTGTAAAGCCTCCGGCAGCTC
CTTTACCGGCTACAACATGAACTGGGTGAGGCAGAACATTGGAAAGTCTTTAGAGTGGATCGGCGCCATC
GATCCCTACTACGGCGGCACCTCCTATAACCAGAAGTTCAAGGGTCGTGCCACCCTCACCGTCGACAAGT
CCTCCAGCACAGCTTATATGCATTTAAAGTCCCTCACCAGCGAGGACTCCGCCGTGTATTATTGTGTGAG
CGGCATGGAATATTGGGGCCAAGGAACCAGCGTCACCGTGTCCAGCGCTTCCACAAAGGGACCCAGCGTC
TTCCCTCTGGCCCCCAGCAGCAAAAGCACCAGCGGCGGAACCGCTGCTTTAGGATGTTTAGTGAAGGACT
ACTTTCCCGAACCCGTGACCGTGAGCTGGAATTCCGGCGCTCTCACCAGCGGCGTGCACACCTTTCCCGC
TGTCCTCCAGTCCAGCGGCCTCTACTCTTTATCCTCCGTGGTGACAGTGCCTAGCTCCTCCCTCGGCACC
CAGACCTACATTTGTAACGTGAACCACAAGCCTTCCAATACAAAGGTGGACAAGAGGGTGGAGCCCAAAA
GCTGCGATAAGACCCACACTCCTCGTAAGATTGAGGTCATGTACCCCCCCCCCTATCTGGACAACGAGAA
GAGCAACGGCACAATCATCCACGTGAAGGGCAAGCATCTGTGCCCCTCCCCTTTATTTCCCGGACCTTCC
AAACCCTTTTGGGTTTTAGTGGTCGTGGGAGGCGTGCTGGCTTGTTACTCTTTACTGGTGACAGTCGCCT
TCATCATCTTTTGGGTGAAGAGGGGTCGTAAGAAACTGCTGTACATCTTCAAACAGCCCTTCATGAGGCC
CGTGCAGACAACCCAAGAAGAGGATGGATGTTCTTGTCGTTTCCCCGAAGAGGAGGAGGGAGGCTGTGAA
CTGAGGGTGAAGTTCTCTCGTAGCGCTGACGCCCCCGCCTACCAACAAGGTCAAAACCAGTTATACAACG
AGCTGAATTTAGGAAGGAGAGAGGAGTATGACGTTTTAGACAAGAGAAGGGGAAGAGACCCCGAAATGGG
CGGCAAGCCCAGAAGGAAGAACCCCCAAGAAGGTTTATACAATGAGCTCCAGAAGGACAAGATGGCCGAG
GCCTACAGCGAAATTGGCATGAAAGGCGAGAGGAGGAGGGGAAAGGGCCATGACGGCCTCTATCAAGGTT
TAAGCACCGCCACCAAGGACACCTACGACGCTTTACATATGCAAGCTTTACCTCCTAGGGGAAGCGGAGA
AGGAAGGGGATCTTTACTGACTTGTGGCGACGTGGAGGAGAACCCCGGCCCTATGTCCGTGCCTACCCAA
GTTCTGGGTTTACTGCTGCTCTGGCTGACAGACGCTCGTTGCGAGATCGTGATGACCCAATCCCCCGCTA
CACTCTCCGTGAGCCCCGGAGAGAGGGCTACTTTAAGCTGTCGTTCCTCCCAGAGCCTCGTCCACAGAAA
CGGCAATACCTATTTACACTGGTATTTACAGAAGCCCGGTCAGAGCCCCAAGTTATTAATTCACAAAGTC
AGCAACAGATTCAGCGGCGTGCCCGATCGTTTCAGCGGCTCCGGCAGCGGAACCGACTTTACACTCAAGA
TCAGCAGAGTGGAGGCCGAGGATCTGGGCGTGTACTTCTGCTCCCAGTCCACCCATGTGCCCCCTCTCAC
ATTCGGAGCCGGAACCAAGCTGGAGCTGAAAAGAACAGTCGCCGCCCCCTCCGTCTTCATTTTTCCCCCT
TCCGATGAGCAGCTGAAGAGCGGCACAGCCAGCGTGGTCTGTCTGCTCAACAATTTTTACCCTCGTGAGG
CCAAGGTCCAGTGGAAGGTGGATAATGCTCTGCAGTCCGGCAACTCCCAAGAATCCGTGACAGAGCAAGA
CTCCAAGGATTCCACCTATTCTTTATCCTCCACCCTCACTTTAAGCAAGGCTGACTACGAGAAGCATAAG
GTGTACGCTTGTGAAGTGACACACCAAGGTTTATCCAGCCCCGTGACAAAGAGCTTCAATAGGGGAGAGT
GC
1^{st} polypeptide, GD2-14.18 DAR,
SEQ ID NO: 32
EVQLLQSGPELEKPGASVMISCKASGSSFTGYNMNWVRQNIGKSLEWIGAIDPYYGGTSYNQKEKGRATL
TVDKSSSTAYMHLKSLTSEDSAVYYCVSGMEYWGQGTSVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKR
VEPKSCDKTHTPRKIEVMYPPPYLDNEKSNGTIIHVKGKHLCPSPLFPGPSKPFWVLVVVGGVLACYSLL
VTVAFIIFWVKRGRKKLLYIFKQPFMRPVQTTQEEDGCSCRFPEEEEGGCELRVKESRSADAPAYQQGON
QLYNELNLGRREEYDVLDKRRGRDPEMGGKPRRKNPQEGLYNELQKDKMAEAYSEIGMKGERRRGKGHDG
LYQGLSTATKDTYDALHMQALPPR
2^{nd} polypeptide, GD2-14.18 DAR,
SEQ ID NO: 33
EIVMTQSPATLSVSPGERATLSCRSSQSLVHRNGNTYLHWYLQKPGQSPKLLIHKVSNRFSGVPDRESGS
GSGTDFTLKISRVEAEDLGVYFCSQSTHVPPLTFGAGTKLELKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSP
VTKSENRGEC
Precursor: GD2-hu14.18 DAR:
SEQ ID NO: 34
MEWSWVFLFFLSVTTGVHSEVQLVQSGAEVEKPGASVKISCKASGSSFTGYNMNWVRQNIGKSLEWIGAI
DPYYGGTSYNQKFKGRATLTVDKSTSTAYMHLKSLRSEDTAVYYCVSGMEYWGQGTSVTVSSASTKGPSV
FPLAPSSKSTSGGTAALGCLVKDYFPEPVTVSWNSGALTSGVHTFPAVLQSSGLYSLSSVVTVPSSSLGT
QTYICNVNHKPSNTKVDKRVEPKSCDKTHTKIEVMYPPPYLDNEKSNGTIIHVKGKHLCPSPLFPGPSKP
FWVLVVVGGVLACYSLLVTVAFIIFWVKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPRGSGEGRGSLLTCGDVEENPGPMSVPTQVL
GLLLLWLTDARCDVVMTQTPLSLPVTPGEPASISCRSSQSLVHRNGNTYLHWYLQKPGQSPKLLIHKVSN
RFSGVPDRESGSGSGTDETLKISRVEAEDLGVYFCSQSTHVPPLTFGAGTKLELKRTVAAPSVFIFPPSD
EQLKSGTASVVCLLNNFYPREAKVQWKVDNALQSGNSQESVTEQDSKDSTYSLSSTLTLSKADYEKHKVY
ACEVTHQGLSSPVTKSENRGEC
Nucleic Acid sequence, GD2-hu14.18 DAR precursor,
SEQ ID NO: 35
atggaatggagctgggtctttctcttcttcctgtcagtaaccaccggtgtccactccGAAGTGCAACTCG
TTCAAAGCGGAGCCGAGGTCGAAAAACCCGGAGCCTCAGTCAAGATTAGCTGTAAAGCTAGTGGCAGCAG
CTTTACAGGGTACAACATGAACTGGGTCCGACAGAACATTGGGAAGTCACTCGAGTGGATTGGGGCTATT
GACCCGTACTACGGTGGAACATCATACAATCAGAAATTTAAAGGACGAGCAACCCTCACGGTAGACAAAA
GTACTTCCACGGCGTATATGCATCTCAAGAGTCTCAGAAGTGAAGACACAGCGGTCTATTATTGTGTATC
TGGTATGGAGTATTGGGGCCAAGGTACGTCCGTCACTGTTTCATCTGCTAGCACAAAAGGCCCCTCCGTC
TTTCCTTTAGCCCCTAGCTCCAAATCCACCTCCGGCGGAACAGCCGCCCTCGGATGTCTCGTCAAAGACT
ACTTCCCCGAGCCCGTGACAGTGTCTTGGAATTCCGGCGCTTTAACCTCCGGAGTGCACACCTTTCCCGC
TGTTTTACAGTCCAGCGGACTGTATTCTTTAAGCTCCGTCGTCACCGTGCCTAGCAGCTCTTTAGGCACC
CAGACCTACATTTGCAACGTCAACCACAAGCCTAGCAACACCAAGGTGGACAAAAGGGTGGAGCCTAAGT
CTTGTGACAAGACCCACACCAAGATCGAGGTCATGTACCCCCCCCCCTATTTAGACAACGAGAAAAGCAA
CGGCACAATTATCCATGTGAAAGGCAAGCATCTCTGCCCCTCCCCTCTGTTTCCCGGACCCTCCAAGCCT
TTTTGGGTGCTCGTGGTGGTGGGCGGCGTGCTGGCTTGTTATTCTTTACTGGTGACCGTCGCCTTTATCA
TCTTCTGGGTGAAGAGGGGTCGTAAGAAGCTGCTGTACATCTTCAAGCAGCCCTTCATGAGGCCCGTTCA
AACCACCCAAGAAGAGGACGGCTGCAGCTGTCGTTTCCCCGAGGAGGAAGAGGGAGGATGCGAGCTGAGG
GTGAAGTTCTCTCGTAGCGCCGATGCCCCCGCTTATCAGCAAGGTCAGAACCAGTTATACAACGAGCTGA
ATTTAGGTCGTAGGGAGGAGTACGACGTGCTGGACAAGAGAAGAGGTCGTGACCCCGAGATGGGCGGCAA
ACCTCGTAGGAAGAACCCCCAAGAAGGTTTATACAATGAGCTGCAGAAGGACAAGATGGCCGAGGCCTAC
AGCGAGATCGGCATGAAGGGCGAGAGGAGAAGGGGCAAGGGCCACGATGGTTTATACCAAGGTTTAAGCA
CCGCCACCAAGGACACCTACGATGCTTTACACATGCAAGCTTTACCTCCTCGTGGAAGCGGCGAAGGAAG
GGGCTCTTTACTGACATGTGGCGACGTGGAGGAAAACCCCGGCCCTATGTCCGTGCCCACACAAGTTCTC
GGACTGTTATTACTGTGGCTGACAGATGCTCGTTGCGATGTCGTTATGACGCAAACCCCGCTTTCCTTGC
CGGTTACGCCTGGAGAACCAGCTTCCATCTCATGTCGGTCTTCACAATCTCTGGTTCATCGCAACGGAAA
CACGTATCTTCATTGGTATCTTCAGAAGCCAGGCCAATCCCCAAAGCTCCTCATTCACAAGGTCTCTAAC
AGATTCAGTGGGGTTCCTGACAGATTTTCCGGGTCTGGATCAGGTACTGACTTCACATTGAAAATTAGCC
GCGTAGAAGCTGAGGACCTGGGCGTATACTTTTGTTCTCAATCCACCCATGTACCACCTCTCACTTTCGG
CGCAGGAACGAAACTGGAACTGAAGAGAACCGTGGCCGCCCCTAGCGTTTTCATTTTCCCCCCCAGCGAC
GAACAGCTGAAGTCCGGCACAGCCTCCGTGGTGTGTTTACTGAATAACTTCTACCCTCGTGAGGCTAAGG
TCCAATGGAAGGTGGATAATGCTTTACAGTCCGGAAATTCCCAAGAAAGCGTGACCGAGCAAGATAGCAA
AGATAGCACCTACAGCTTAAGCTCCACACTGACACTGTCCAAAGCCGACTACGAGAAACACAAGGTGTAC
GCTTGTGAAGTGACCCATCAAGGTTTAAGCTCCCCCGTCACAAAGAGCTTTAACAGAGGAGAATGC
1^{st} polypeptide, GD2-hu14.18 DAR
SEQ ID NO: 36
EVQLVQSGAEVEKPGASVKISCKASGSSFTGYNMNWVRQNIGKSLEWIGAIDPYYGGTSYNQKFKGRATL
TVDKSTSTAYMHLKSLRSEDTAVYYCVSGMEYWGQGTSVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKR
VEPKSCDKTHTKIEVMYPPPYLDNEKSNGTIIHVKGKHLCPSPLFPGPSKPFWVLVVVGGVLACYSLLVT
VAFIIFWVKRGRKKLLYIFKQPFMRPVQTTQEEDGCSCRFPEEEEGGCELRVKFSRSADAPAYQQGONQL
YNELNLGRREEYDVLDKRRGRDPEMGGKPRRKNPQEGLYNELQKDKMAEAYSEIGMKGERRRGKGHDGLY
QGLSTATKDTYDALHMQALPPR
2^{nd} polypeptide, GD2-hu14.18 DAR
SEQ ID NO: 37
DVVMTQTPLSLPVTPGEPASISCRSSQSLVHRNGNTYLHWYLQKPGQSPKLLIHKVSNRFSGVPDRESGS
GSGTDFTLKISRVEAEDLGVYFCSQSTHVPPLTFGAGTKLELKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSP
VTKSENRGEC
G4S linker
SEQ ID NO: 38
GGGGSGGGGSGGGGS
JeT promoter
SEQ ID NO: 39
GAATTCGGGCGGAGTTAGGGCGGAGCCAATCAGCGTGCGCCGTTCCGAAAGTTGCC
TTTTATGGCTGGGCGGAGAATGGGCGGTGAACGCCGATGATTATATAAGGACGCGC
CGGGTGTGGCACAGCTAGTTCCGTCGCAGCCGGGATTTGGGTCGCGGTTCTTGTTTG
TGGATCCCTGTGATCGTCAGTTGACA
DNA
Homo sapiens
5′ homology arm from exon 1 of TRAC gene, Cas9 target site, 660 nt
SEQ ID NO: 40
GGCACCATATTCATTTTGCAGGTGAAATTCCTGAGATGTAAGGAGCTGCTGTGACTTGCTCAAG
GCCTTATATCGAGTAAACGGTAGTGCTGGGGCTTAGACGCAGGTGTTCTGATTTATAGTTCAAA
ACCTCTATCAATGAGAGAGCAATCTCCTGGTAATGTGATAGATTTCCCAACTTAATGCCAACAT
ACCATAAACCTCCCATTCTGCTAATGCCCAGCCTAAGTTGGGGAGACCACTCCAGATTCCAAGA
TGTACAGTTTGCTTTGCTGGGCCTTTTTCCCATGCCTGCCTTTACTCTGCCAGAGTTATATTGC
TGGGGTTTTGAAGAAGATCCTATTAAATAAAAGAATAAGCAGTATTATTAAGTAGCCCTGCATT
TCAGGTTTCCTTGAGTGGCAGGCCAGGCCTGGCCGTGAACGTTCACTGAAATCATGGCCTCTTG
GCCAAGATTGATAGCTTGTGCCTGTCCCTGAGTCCCAGTCCATCACGAGCAGCTGGTTTCTAAG
ATGCTATTTCCCGTATAAAGCATGAGACCGTGACTTGCCAGCCCCACAGAGCCCCGCCCTTGTC
CATCACTGGCATCTGGACTCCAGCCTGGGTTGGGGCAAAGAGGGAAATGAGATCATGTCCTAAC
CCTGATCCTCTTGTCCCACA
DNA
Homo sapiens
3′ homology arm from exon 1 of TRAC gene, Cas9 target site, 650 nt
SEQ ID NO: 41
GATATCCAGAACCCTGACCCTGCCGTGTACCAGCTGAGAGACTCTAAATCCAGTGACAAGTCTG
TCTGCCTATTCACCGATTTTGATTCTCAAACAAATGTGTCACAAAGTAAGGATTCTGATGTGTA
TATCACAGACAAAACTGTGCTAGACATGAGGTCTATGGACTTCAAGAGCAACAGTGCTGTGGCC
TGGAGCAACAAATCTGACTTTGCATGTGCAAACGCCTTCAACAACAGCATTATTCCAGAGGACA
CCTTCTTCCCCAGCCCAGGTAAGGGCAGCTTTGGTGCCTTCGCAGGCTGTTTCCTTGCTTCAGG
AATGGCCAGGTTCTGCCCAGAGCTCTGGTCAATGATGTCTAAAACTCCTCTGATTGGTGGTCTC
GGCCTTATCCATTGCCACCAAAACCCTCTTTTTACTAAGAAACAGTGAGCCTTGTTCTGGCAGT
CCAGAGAATGACACGGGAAAAAAGCAGATGAAGAGAAGGTGGCAGGAGAGGGCACGTGGCCCAG
CCTCAGTCTCTCCAACTGAGTTCCTGCCTGCCTGCCTTTGCTCAGACTGTTTGCCCCTTACTGC
TCTTCTAGGCCTCATTCTAAGCCCCTTCTCCAAGTTGCCTCTCCTTATTTCTCCCTGTCTGCCA
AAAAATCTTT
DNA
Homo sapiens
Cas9 target site , TRAC locus
SEQ ID NO: 42
CAGGGTTCTGGATATCTGT
DNA
Artificial
Forward primer
SEQ ID NO: 43
A*TmC*mA*mCGAGCAGCTGGTTTCT
(*indicates phosphorothioate linkage;
mA indicates 2′-O-methyladenosine;
mC indicates 2′-O-methylcytidine)
DNA
Artificial
Reverse primer
SEQ ID NO: 44
GACCTCATGTCTAGCACAGTTTTG
DNA
Homo sapiens
171 bp 5′ homology region, Cas9 target site
SEQ ID NO: 45
ATCACGAGCAGCTGGTTTCTAAGATGCTATTTCCCGTATAAAGCATGAGACCGTGACTTGCCAG
CCCCACAGAGCCCCGCCCTTGTCCATCACTGGCATCTGGACTCCAGCCTGGGTTGGGGCAAAGA
GGGAAATGAGATCATGTCCTAACCCTGATCCTCTTGTCCCACA
DNA
Homo sapiens
161 bp 3′ homology region, Cas9 target site
SEQ ID NO: 46
GATATCCAGAACCCTGACCCTGCCGTGTACCAGCTGAGAGACTCTAAATCCAGTGACAAGTCTG
TCTGCCTATTCACCGATTTTGATTCTCAAACAAATGTGTCACAAAGTAAGGATTCTGATGTGTA
TATCACAGACAAAACTGTGCTAGACATGAGGTC
IgG1 upper hinge sequence
SEQ ID NO: 47
EPKSCDKTHT
IgG1 core hinge sequence
SEQ ID NO: 48
CPXC, wherein X is P, R or S
lower hinge/CH2 sequence
SEQ ID NO: 49
PAPELLGGP
segment of Fc region (CH2)
SEQ ID NO: 50
SVFLFPPKPKDT
hinge sequence including upper, core and lower hinge
SEQ ID NO: 51
EPKSCDKTHTCPPCPAPELLGGP
SEQ ID NO: 52
LAGLIDADG

We claim:

1. A genetically modified host cell, or a population of genetically modified host cells, expressing a dimeric antigen receptor (DAR) that binds GD2, wherein the DAR comprises:
a. a first polypeptide comprising a plurality of polypeptide regions ordered from the amino terminus to the carboxyl terminus: (i) an antibody heavy chain variable region, (ii) an antibody heavy chain constant region, (iii) a hinge region, (iv) a transmembrane region, and (v) an intracellular region; and
b. a second polypeptide comprising a plurality of polypeptide regions ordered from the amino terminus to the carboxyl terminus: (i) an antibody light chain variable region and (ii) an antibody light chain constant region, wherein the second polypeptide does not comprise a transmembrane region;
wherein the antibody heavy chain constant region and the antibody light chain constant region form a dimerization domain for formation of the DAR, and wherein the antibody heavy chain variable region and the antibody light chain variable region form an antigen binding domain that binds GD2.

2. A genetically modified host cell, or a population of genetically modified host cells, according to claim 1, wherein the DAR comprises:
a. a first polypeptide consisting essentially of: (i) an antibody heavy chain variable region, (ii) an antibody heavy chain constant region, (iii) a hinge region, (iv) a transmembrane region, and (v) an intracellular region; and
b. a second polypeptide consisting essentially of: (i) an antibody light chain variable region and (ii) an antibody light chain constant region.

3. A genetically modified host cell, or a population of genetically modified host cells, expressing a dimeric antigen receptor (DAR) that binds GD2, wherein the DAR comprises:
a) a first polypeptide chain comprising a plurality of polypeptide regions ordered from the amino terminus to the carboxyl terminus: (i) an antibody light chain variable region, (ii) an antibody light chain constant region, (iii) a hinge region, (iv) a transmembrane region, and (v) an intracellular region; and
b) a second polypeptide chain comprising a plurality of polypeptide regions ordered from the amino terminus to the carboxyl terminus: (i) an antibody heavy chain variable region and (ii) an antibody heavy chain constant region, wherein the second polypeptide does not comprise a transmembrane region;
wherein the antibody heavy chain constant region and the antibody light chain constant region form a dimerization domain for formation of the dimeric antigen receptor (DAR), and
wherein the antibody heavy chain variable region and the antibody light chain variable region form an antigen binding domain that binds GD2.

4. A genetically modified host cell, or a population of genetically modified host cells, according to claim 3, wherein the DAR comprises:
a. a first polypeptide consisting essentially of: (i) an antibody light chain variable region, (ii) an antibody light chain constant region, (iii) a hinge region, (iv) a transmembrane region, and (v) an intracellular region; and
b. a second polypeptide consisting of: (i) an antibody heavy chain variable region and (ii) an antibody heavy chain constant region.

5. A genetically modified host cell, or a population of genetically modified host cells, according to any one of claims 1-4, wherein the antibody heavy chain constant region and the antibody light chain constant region dimerize via one or more disulfide bonds.

6. A genetically modified host cell, or a population of genetically modified host cells, according to any one of claims 1-4, wherein the hinge region comprises a hinge sequence from an antibody selected from a group consisting of IgG, IgA, IgM, IgE and IgD.

7. A genetically modified host cell, or a population of genetically modified host cells, according to any one of claims 1-4, wherein the hinge region comprises a CD8a hinge region, a CD28 hinge region, or a CD8 α/CD28 hinge region, or a hinge region having at least 95% identity to any thereof.

8. A genetically modified host cell, or a population of genetically modified host cells, according to claim 7, wherein the hinge comprises a CD28 hinge region (SEQ ID NO:9) or a hinge region having at least 95% identity thereto.

9. A genetically modified host cell, or a population of genetically modified host cells, according to any one of claims 1-4, wherein the transmembrane region comprises a CD8, CD28, 4-1BB, or CD3ζ transmembrane region, or a transmembrane region having at least 95% identity to any thereof.

10. A genetically modified host cell, or a population of genetically modified host cells, according to claim 9, wherein the transmembrane region comprises a transmembrane domain sequence from CD28 (SEQ ID NO: 12) or a sequence having at least 95% identity thereto.

11. A genetically modified host cell, or a population of genetically modified host cells, according to any one of claims 1-4, wherein the intracellular region comprises one or more intracellular amino acid sequences selected from a group consisting of 4-1BB intracellular region (SEQ ID NO:16), CD3ζ having ITAMs 1, 2 and 3 (SEQ ID NO:19), CD3ζ having ITAM 1 (SEQ ID NO:20), CD3ζ having ITAM 3 (SEQ ID NO:22), or an intracellular region of any of CD28 (SEQ ID NO:17), CD27, OX40 (SEQ ID NO:18), CD30, CD40, PD-1, ICOS, lymphocyte function-associated antigen-1 (LFA-1), CD2, CD7, LIGHT, NKG2C, B7-H3, GITR (TNFRSF18), DR3 (TNFRSF25), TNFR2 and/or CD226, or an intracellular amino acid sequence having at least 95% identity to any thereof.

12. A genetically modified host cell, or a population of genetically modified host cells, according to claim 11, wherein the intracellular region comprises a CD35 having ITAMs 1, 2 and 3 (SEQ ID NO:19).

13. A genetically modified host cell, or a population of genetically modified host cells, according to claim 12, wherein the intracellular region further comprises a 4-1BB intracellular region (SEQ ID NO:16).

14. A genetically modified host cell, or a population of genetically modified host cells, according to any one of claims 1-4, wherein the antibody heavy chain variable region comprises an amino acid sequence having at least 95% identity to SEQ ID NO:2.

15. A genetically modified host cell, or a population of genetically modified host cells, according to claim 14, wherein the antibody light chain constant region comprises an amino acid sequence having at least 95% identity to SEQ ID NO:5.

16. A genetically modified host cell, or a population of genetically modified host cells, according to any one of claims 1-4, wherein the antibody heavy chain variable region comprises an amino acid sequence having at least 95% identity to SEQ ID NO:3.

17. A genetically modified host cell, or a population of genetically modified host cells, according to claim 16, wherein the antibody light chain constant region comprises an amino acid sequence having at least 95% identity to SEQ ID NO:6.

18. A genetically modified host cell, or a population of genetically modified host cells, according to claim 1 or claim 2, wherein the first polypeptide chain comprises the amino acid sequence of SEQ ID NO:32.

19. A genetically modified host cell, or a population of genetically modified host cells, according to claim 18, wherein the second polypeptide chain comprises the amino acid sequence of SEQ ID NO:33.

20. A genetically modified host cell, or a population of genetically modified host cells, according to claim 1 or claim 2, wherein the first polypeptide chain comprises the amino acid sequence of SEQ ID NO:36.

21. A genetically modified host cell, or a population of genetically modified host cells, according to claim 20, wherein the second polypeptide chain comprises the amino acid sequence of SEQ ID NO:37.

22. A genetically modified host cell, or a population of genetically modified host cells, according to claim 1 or claim 2, wherein the DAR comprises:
a) a first polypeptide chain comprising or consisting essentially of a plurality of polypeptide regions ordered from the amino terminus to the carboxyl terminus: (i) a GD2 antibody heavy chain variable region comprising an amino acid sequence having at least 95% identity to SEQ ID NO:2; (ii) a GD2 antibody heavy chain constant region comprising an amino acid sequence having at least 95% identity to SEQ ID NO:4; (iii) the hinge region of CD28 (SEQ ID NO:9) or a hinge region having at least 95% identity thereto; (iv) the transmembrane region of CD28 (SEQ ID NO:12) or an transmembrane domain having at least 95% identity thereto; and (v) an intracellular region comprising a 4-1BB intracellular domain (SEQ ID NO:16) and a CD3zeta intracellular region comprising ITAMs 1, 2 and 3 (SEQ ID NO:19); and
b) a second polypeptide chain comprising or consisting essentially of a plurality of polypeptide regions ordered from the amino terminus to the carboxyl terminus: (i) a GD2 antibody light chain variable region comprising an amino acid sequence having at least 95% identity to SEQ ID NO:5; and (ii) a GD2 antibody light chain constant region comprising an amino acid sequence having at least 95% identity to SEQ ID NO:7, wherein the second polypeptide chain does not comprise a transmembrane domain.

23. A genetically modified host cell, or a population of genetically modified host cells, according to claim 1 or claim 2, wherein
a) the first polypeptide chain comprises or consists essentially of a plurality of polypeptide regions ordered from the amino terminus to the carboxyl terminus: (i) a GD2 antibody heavy chain variable region comprising an amino acid sequence having at least 95% identity to SEQ ID NO:3; (ii) a GD2 antibody heavy chain constant region comprising an amino acid sequence having at least 95% identity to SEQ ID NO:4; (iii) the hinge region of CD28 (SEQ ID NO:9) or a hinge region having at least 95% identity thereto; (iv) the transmembrane region of CD28 (SEQ ID NO:12) or an transmembrane domain having at least 95% identity thereto; and (v) an intracellular region comprising a 4-1BB intracellular domain (SEQ ID NO:16) and a CD3zeta intracellular region comprising ITAMs 1, 2 and 3 (SEQ ID NO:19); and
b) the second polypeptide chain comprises or consists essentially of a plurality of polypeptide regions ordered from the amino terminus to the carboxyl terminus: (i) a GD2 antibody light chain variable region comprising an amino acid sequence having at least 95% identity to SEQ ID NO:6; and (ii) a GD2 antibody light chain constant region comprising an amino acid sequence having at least 95% identity to SEQ ID NO:7, wherein the second polypeptide chain does not comprise a transmembrane domain.

24. A genetically modified host cell, or a population of genetically modified host cells, according to claim 3 or claim 4, wherein the DAR comprises:
(a) a first polypeptide chain comprising or consisting essentially of a plurality of polypeptide regions ordered from the amino terminus to the carboxyl terminus: (i) a GD2 antibody light chain variable region comprising an amino acid sequence having at least 95% identity to SEQ ID NO:5; (ii) a GD2 antibody light chain constant region comprising an amino acid sequence having at least 95% identity to SEQ ID NO:7;
(iii) the hinge region of CD28 (SEQ ID NO:9) or a hinge region having at least 95% identity thereto; (iv) the transmembrane region of CD28 (SEQ ID NO:12) or an transmembrane domain having at least 95% identity thereto; and (v) an intracellular region comprising a 4-1BB intracellular domain (SEQ ID NO:16) and a CD3zeta intracellular region comprising ITAMs 1, 2 and 3 (SEQ ID NO:19); and
(b) a second polypeptide chain comprising or consisting essentially of a plurality of polypeptide regions ordered from the amino terminus to the carboxyl terminus: (i) a GD2 antibody heavy chain variable region comprising an amino acid sequence having at least 95% identity to SEQ ID NO:2; and (ii) a GD2 antibody heavy chain constant region comprising an amino acid sequence having at least 95% identity to SEQ ID NO:4, wherein the second polypeptide chain does not comprise a transmembrane domain.

25. A genetically modified host cell, or a population of genetically modified host cells, according to claim 1 or claim 2, wherein
(a) the first polypeptide chain comprises or consists essentially of a plurality of polypeptide regions ordered from the amino terminus to the carboxyl terminus: (i) a GD2 antibody light chain variable region comprising an amino acid sequence having at least 95% identity to SEQ ID NO:6; (ii) a GD2 antibody light chain constant region comprising an amino acid sequence having at least 95% identity to SEQ ID NO:7; (iii) the hinge region of CD28 (SEQ ID NO:9) or a hinge region having at least 95% identity thereto; (iv) the transmembrane region of CD28 (SEQ ID NO:12) or an transmembrane domain having at least 95% identity thereto; and (v) an intracellular region comprising a 4-1BB intracellular domain (SEQ ID NO:16) and a CD3zeta intracellular region comprising ITAMs 1, 2 and 3 (SEQ ID NO:19); and
(b) the second polypeptide chain comprises or consists essentially of a plurality of polypeptide regions ordered from the amino terminus to the carboxyl terminus: (i) a GD2 antibody heavy chain variable region comprising an amino acid sequence having at least 95% identity to SEQ ID NO:3; and (ii) a GD2 antibody heavy chain constant region comprising an amino acid sequence having at least 95% identity to SEQ ID NO:4, wherein the second polypeptide chain does not comprise a transmembrane domain.

26. A genetically modified host cell, or a population of genetically modified host cells, according to claim 22 or claim 23, wherein (a) the first polypeptide chain comprises (i) a GD2 antibody heavy chain variable region comprising the amino acid sequence of SEQ ID NO:2; and b) the second polypeptide chain comprises (i) a GD2 antibody light chain variable region comprising the amino acid sequence of SEQ ID NO:5.

27. A genetically modified host cell, or a population of genetically modified host cells, according to claim 22 or claim 23, wherein (a) the first polypeptide chain comprises (i) a GD2 antibody heavy chain variable region comprising the amino acid sequence of SEQ ID NO:3; and b) the second polypeptide chain comprises (i) a GD2 antibody light chain variable region comprising the amino acid sequence of SEQ ID NO:6.

28. A genetically modified host cell, or a population of genetically modified host cells, according to any of claims 1-27, wherein the genetically modified host cell or population of genetically modified host cells comprises at least one nucleic acid sequence encoding the first polypeptide and the second polypeptide.

29. A genetically modified host cell, or a population of genetically modified host cells, according to claim 28, wherein at least one nucleic acid sequence encodes the polypeptide of SEQ ID NO:32 and the polypeptide of SEQ ID NO:33.

30. A genetically modified host cell, or a population of genetically modified host cells, according to claim 29, wherein at least one nucleic acid sequence encodes a precursor polypeptide of SEQ ID NO:30.

31. A genetically modified host cell, or a population of genetically modified host cells, according to claim 30, wherein at least one nucleic acid sequence comprises a SEQ ID NO:31 or a sequence having at least 65% identity thereto.

32. A genetically modified host cell, or a population of genetically modified host cells, according to claim 28, wherein at least one nucleic acid sequence encodes the polypeptide of SEQ ID NO:36 and the polypeptide of SEQ ID NO:37.

33. A genetically modified host cell, or a population of genetically modified host cells, according to claim 32, wherein at least one nucleic acid sequence encodes a precursor polypeptide of SEQ ID NO:34.

34. A genetically modified host cell, or a population of genetically modified host cells, according to claim 33, wherein at least one nucleic acid sequence comprises a SEQ ID NO:35 or a sequence having at least 65% identity thereto.

35. A genetically modified host cell, or a population of genetically modified host cells, according to any of claims 1-34, comprising T lymphocytes, NK (natural killer) cells, macrophages, dendritic cells, mast cells, eosinophils, B lymphocytes or monocytes.

36. A population of host cells of claim 35, wherein the cells are primary cells.

37. A population of host cells of claim 35, wherein the cells are human cells.

38. A population of host cells according to claim 35, wherein the population comprises T cells.

39. A population of host cells according to claim 35, wherein at least 90% of the cells that express the DAR do not express the T cell receptor.

40. A population of host cells according to claim 35, wherein at least 95% of the cells that express the DAR do not express the T cell receptor.

41. A pharmaceutical composition comprising a pharmaceutically-acceptable excipient and a population of host cells according to any of claims 35-40.

42. The pharmaceutical composition of claim 41, provided in a bag, vial, tube, or dish.

43. The pharmaceutical composition of claim 41, wherein the composition is frozen.

44. A method for treating a subject having cancer, comprising administering to the subject the population of host cells of any of claims 1-40 or the pharmaceutical composition of any of claims 41-43.

45. The method of claim 44, wherein the cancer is breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, a leiomyoma, a leiomyosarcoma, a glioma, a glioblastoma, a neuroblastomas, small-cell lung cancer, a medulloblastoma, an astrocytoms, or an osteosarcoma.

46. The method of claim 44, wherein the cancer is melanoma or osteosarcoma.

47. The method of any of claims 44-46, wherein the population of cells is administered multiple times over a period of days, weeks, or months.

48. At least one nucleic acid molecule encoding:
a) a first polypeptide comprising a plurality of polypeptide regions ordered from the amino terminus to the carboxyl terminus: (i) an antibody heavy chain variable region, (ii) an antibody heavy chain constant region, (iii) a transmembrane region, and (iv) an intracellular region; and
b) a second polypeptide comprising a plurality of polypeptide regions ordered from the amino terminus to the carboxyl terminus: (i) an antibody light chain variable region and (ii) an antibody light chain constant region, wherein the second polypeptide does not include a transmembrane domain;
wherein the antibody heavy chain constant region and the antibody light chain constant region form a dimerization domain for formation of the DAR, and wherein the antibody heavy chain variable region and the antibody light chain variable region form an antigen binding domain that binds GD2.

49. At least one nucleic acid molecule encoding:
a) a first polypeptide chain comprising a plurality of polypeptide regions ordered from the amino terminus to the carboxyl terminus: (i) an antibody light chain variable region, (ii) an antibody light chain constant region, (iii) a transmembrane region, and (iv) an intracellular region; and
b) a second polypeptide chain comprising a plurality of polypeptide regions ordered from the amino terminus to the carboxyl terminus: (i) an antibody heavy chain variable region and (ii) an antibody heavy chain constant region, wherein the second polypeptide does not include a transmembrane domain;
wherein the antibody heavy chain constant region and the antibody light chain constant region form a dimerization domain for formation of the dimeric antigen receptor (DAR), and
wherein the antibody heavy chain variable region and the antibody light chain variable region form an antigen binding domain that binds GD2.

50. A nucleic acid molecule encoding a precursor polypeptide comprising a plurality of polypeptide regions ordered from the amino terminus to the carboxyl terminus: (1) a heavy chain leader sequence (2) an antibody heavy chain variable region, (3) an antibody heavy chain constant region, (4) an optional hinge region, (5) a transmembrane region, (6) an intracellular region, (7) a self-cleaving sequence, (8) a light chain leader sequence, (9) an antibody light chain variable region, and (10) an antibody light chain constant region, wherein the self-cleaving sequence permits cleaving the of the precursor polypeptide into a first and second polypeptide chain.

51. A nucleic acid molecule encoding a precursor polypeptide comprising a plurality of polypeptide regions ordered from the amino terminus to the carboxyl terminus: (1) a light chain leader sequence (2) an antibody light chain variable region, (3) an antibody light chain constant region, (4) an optional hinge region, (5) a transmembrane region, (6) an intracellular region, (7) a self-cleaving sequence, (8) a heavy chain leader sequence, (9) an antibody heavy chain variable region, and (10) an antibody heavy chain constant region, wherein the self-cleaving sequence permits cleaving the of the precursor polypeptide into a first and second polypeptide chain.

52. The one or more nucleic acid molecules of any one of claims 48-51, wherein the antibody heavy chain variable region comprises the amino acid sequence of SEQ ID NO:2.

53. The one or more nucleic acid molecules of any one of claims 48-52, wherein the antibody heavy chain constant region comprises the amino acid sequence of SEQ ID NO:3.

54. The one or more nucleic acid molecules of any one of claims 48-53, wherein the antibody light chain variable region comprises the amino acid sequence of SEQ ID NO:5.

55. The one or more nucleic acid molecules of any one of claims 48-54, wherein the antibody light chain constant region comprises the amino acid sequence of SEQ ID NO:6.

56. The nucleic acid molecule of claim 50, comprising the amino acid sequence of SEQ ID NO:31 or SEQ ID NO:35.
